(19) United States
(12) Patent Application Publication
(10) Pub. No.: US 2024/0170146 A1
(43) Pub. Date: May 23, 2024

(54) SYSTEM TO IDENTIFY TRANSITIONS IN BRAIN STATES FROM ELECTROPHYSIOLOGICAL MARKERS IN DBS FOR DEPRESSION

(71) Applicants: ICAHN SCHOOL OF MEDICINE AT MOUNT SINAI, New York, NY (US); GEORGIA TECH RESEARCH CORPORATIONS, Atlanta, GA (US); EMORY UNIVERSITY, Atlanta, GA (US)

(72) Inventors: Sankaraleengam Alagapan, Atlanta, GA (US); Christopher Rozell, Atlanta, GA (US); Helen Mayberg, New York, NY (US); Vineet Tiruvadi, Atlanta, GA (US); Allison Waters, New York, NY (US); Patricio Riva-Posse, Atlanta, GA (US); Stephen Heisig, New York, NY (US); Robert Butera, Atlanta, GA (US); Ki Sueng Choi, New York, NY (US); Andrea Crowell, Atlanta, GA (US)

(21) Appl. No.: 18/213,070

(22) Filed: Jun. 22, 2023

Related U.S. Application Data
(63) Continuation-in-part of application No. PCT/US2022/045422, filed on Sep. 30, 2022.
(60) Provisional application No. 63/228,785, filed on Aug. 3, 2021.

(51) Int. Cl. G16H 50/20; G16H 10/60; G16H 50/50

(52) U.S. Cl. CPC G16H 50/20; G16H 10/60; G16H 50/50

(57) ABSTRACT

Methods and systems are described for providing deep brain stimulation (DBS) of the subcallosal cingulate region (SCC) to provide symptom relief in patients with treatment-resistant depression (TRD). The DBS devices may enable chronic electrophysiology recording to deliver SCC DBS therapy. One or more machine-learning models may identify SCC LFP changes that indicate the patient's current clinical state. The clinical state is distinct from acute transient stimulation effects, is sensitive to DBS stimulation adjustments, and accurately captures individual differences in recovery trajectory.

### CROSS REFERENCE TO RELATED APPLICATIONS FILED

This application is a continuation-in-part of Patent Cooperation Treaty Application No. PCT/US2022/045422, filed Sep. 30, 2022, which claims the priority benefit of U.S. Provisional Patent Application No. 62/228,785, filed Aug. 3, 2021, the disclosures of which are incorporated by reference herein in their entirety for all purposes.

### BACKGROUND

Major depression is a debilitating disorder characterized by the persistence of a depressed mood and other symptoms over many days. Deep brain stimulation (DBS) of the subcallosal cingulate cortex (SCC) has been demonstrated to be effective in treating patients experiencing treatment-resistant depression (TRD), While inconclusive results were reported in a multicenter double-blind clinical trial targeting the SCC gray matter, refinement of the stimulation target to tractography-defined white matter tracts in the ventromedial prefrontal cortex has improved patient outcomes with response rates approaching 90% in open-label studies. Understanding how DBS influences the neural dynamics that underlie this response will help improve this therapy by allowing treatment decisions to be made based on objective physiological changes rather than behavioral observations that may be confounded by short-term mood variations. However, due to the infeasibility of chronic SCC recording during therapy, there is limited knowledge of the neurophysiological changes over long timescales (order of weeks) that underlie stable recovery, where symptom relief is consistent from week to week). While the current understanding of SCC dynamics arises from acute recordings of local field potentials, it is unclear how this may relate to changes due to chronic stimulation.

Recent advances in neurotechnologies have enabled platforms for long-term monitoring of electrophysiological dynamics with the aim of closed-loop stimulation using implanted devices. These platforms are used in the treatment of neurological disorders like Parkinson's disease, essential tremor, and epilepsy. This enhanced capability has led to the collection of large amounts of data spanning different modalities (e.g., electrophysiology, imaging, actimetry, self-reports, and video recordings of behavior) and the application of machine learning techniques to provide insight from this multi-modal, multi-dimensional data. Machine-learning methods have been used with neurophysiological features to distinguish patients with depression from healthy controls, identify subtypes of depression, and predict treatment outcomes. In conventional machine learning approaches, there is typically a tradeoff between complexity and interpretability: simple models can be interpretable but capture only rudimentary structure in the data, while more complex ‘black-box’ models can capture more complex relationships at the expense of interpretability. Earlier studies utilizing machine learning techniques often used simpler models, prioritizing interpretability over model complexity.

### SUMMARY

In one aspect, the disclosure provides a method or system to assess major depressive disorder (MDD) disease state in a subject during the course of therapy, the method or system including the use of electrophysiological measurements for assessment.

In another aspect, the disclosure provides a method or system to characterize the progression of MDD in a subject during the course of therapy, the method or system comprising the use of chronic changes in electrophysiology measurements from the brain to characterize the progression. In some aspects, the characterization comprises the identification of at least one discrete disease state or the disease trajectory within at least one disease state.

In one aspect, the disclosure provides the use of chronic electrophysiology signals as a biomarkers to assess MDD disease state in a subject during the course of therapy, characterize the progression of MDD in a subject during the course of therapy, and/or monitor, characterize, and/or assess discrete transitions in behavior during the course of therapy.

In some aspects, the therapy comprises neural stimulation. In another aspects, the neural stimulation is acute.

In an aspect, the disclosure provides a method to track changes in facial feature between discrete disease states.

In another aspect, the disclosure provides a structural connectivity brain map for predicting when transitions in brain states may occur in a subject in need thereof.

These and other advantages, aspects, and novel features of the present disclosure, as well as details of an illustrated embodiment thereof, will be more fully understood from the following description and drawings.

### BRIEF DESCRIPTION OF THE DRAWINGS

Illustrative embodiments of the present application, including its systems and methods, are summarized below in the following drawing figures:

FIG. 1A illustrates an axial view of the DBS lead targeting bilateral SCC in an example patient according to aspects of the present disclosure. The sphere indicate the volume of tissue activated (VTA) with the final stimulation intensity. The circles indicate the volume of tissue each electrode contact records (VTR) from, showing coverage of grey matter which are the likely sources of the recorded LFP.

FIG. 1B illustrates common structural connectivity patterns from chronic stimulation VTA seed of the six patients at six months. CB—Cingulum Bundle, OF—Uncinate Fasciculus, FM—Forceps Minor, F-ST—Frontostriatal fibers, according to aspects of the present disclosure.

FIG. 1C illustrates an exemplary trajectory of symptoms for six patients according to aspects of the present disclosure. Red line indicates the relapsed responder and grey lines indicate the other 5 typical participants. Black line indicates the average of the 5 typical participants. Error bars indicate standard deviation.

FIG. 1D illustrates an exemplary schematic of inferring spectral discriminative components (SDCs) from LFP features according to aspects of the present disclosure. The neural network may be trained with the data from the ‘sick’ and ‘well’ states. A general causal explainer (GCE) may be trained with the data from the ‘sick’ and ‘well’ states. The data from the intermediate period can be projected through the trained GCE to obtain the SDCs.

FIG. 1E illustrates a framework for analyzing a black-box classifier using generative causal explainer (GCE) according to aspects of the present disclosure. A generative model may transform data from a high dimensional feature space to low dimensional latent space such that at least one of the components may represent the difference between a set of classes as captured by the classifier (discriminative dimension). Other dimensions may capture variance in data not associated with difference between the classes.

FIG. 1F illustrates an example transition identification procedure according to aspects of the present disclosure. Transition to a stable response may be defined as the week when the measure of interest falls below the transition threshold and remains below the threshold for three consecutive weeks.

FIG. 2A illustrates an example coronal view of the DBS lead targeting bilateral SCC in an example patient according to aspects of the present disclosure. The red sphere indicates the volume of tissue activated (VTA) with the final stimulation parameters. The black circles indicate the volume of tissue recorded (VTR) from each electrode contact, showing coverage of grey matter which are the likely sources of the recorded LFP.

FIG. 2B illustrates example common activation pathway patterns from chronic stimulation VTA seed of the 6 participants at six months. CB—Cingulum Bundle, UF—Uncinate Fasciculus, FM—Forceps Minor, F-ST—Frontostriatal fibers according to aspects of the present disclosure.

FIG. 2C illustrates example trajectory of HDRS-17 scores over 24 weeks for five participants (of six total) who were typical responders according to aspects of the present disclosure. Grey lines indicate individuals and black line indicates the average. Error bars indicate standard deviation. Clinical consensus was all five were ‘sick’ during weeks 1-4 and in ‘stable response’ during weeks 20-24.

FIG. 2D illustrates an example schematic for deriving the spectral discriminative component (SDC) from LFP features according to aspects of the present disclosure. A neural network classifier can be first trained with data from the ‘sick’ and ‘stable response’ states of all typical responders. Separate neural networks can be trained to compress the data from the spectral feature space to a low-dimensional latent space and then reconstruct the data from that compressed version. One of these latent dimensions may be a discriminative component constrained to represent the simultaneous data changes (the SDC) used by the classifier to distinguish ‘sick’ from stable response.

FIG. 2E illustrates an example utility of an objective biomarker according to aspects of the present disclosure. When patients experience instability in symptom scores, decisions about treatment (e.g., stimulation voltage adjustment) must be made by inferring whether the instability is due to transient distress (scenario 1) or depression relapse (scenario 2). A biomarker that reflects progress of the brain toward ‘stable response’ can enable more effective clinical decision making about interventions.

FIG. 3A illustrates example receiver operating characteristic (ROC) curves of a neural network classifier in classifying ‘sick’ and ‘well’ states with leave-one-participant out cross validation according to aspects of the present disclosure. Grey lines indicate the ROC curve of individual folds of the cross validation. Black lines indicate the mean ROC curve.

FIG. 3B illustrates change in feature strength for a unit change in a discriminative component indicating the features with highest difference between ‘sick’ and ‘well’ state according to aspects of the present disclosure. + indicates the top 5 features.

FIG. 3C illustrates an example graph of a trajectory of SDCs for six patients according to aspects of the present disclosure. Red line indicates the relapsed responder and grey lines indicate the other 5 typical participants. Black line indicates the average of the 5 typical participants. Error bars indicate standard deviation.

FIG. 3D illustrates an example graph of a change in left low beta power relative to last week of post-surgical period without stimulation from the beginning of the treatment phase to the end of treatment phase according to aspects of the present disclosure.

FIG. 4A illustrates example receiver operating characteristic (ROC) curves of the LFP classifier in classifying ‘sick’ and ‘stable response’ states with leave-one-participant out cross validation according to aspects of the present disclosure. Grey lines indicate the ROC curve of individual folds of the cross validation. Black line indicates the mean ROC curve.

FIG. 4B illustrates an example simultaneous change in spectral features that capture the difference between the ‘sick’ and ‘stable response’ states as reflected by the SDC. The + symbol indicates the top 5 discriminative features according to aspects of the present disclosure

FIG. 4C illustrates an example change in left low beta and high beta power from the beginning of the observation phase to the end of observation phase (relative to last week of post-surgical period without stimulation). *p<0.05 according to aspects of the present disclosure.

FIG. 4D illustrates an example Trajectory of the SDC over 24 weeks. Grey lines indicate individual participants and black line indicates the average of the five typical responders. Error bars indicate standard deviation.

FIG. 4E illustrates an example of identifying state change from ‘sick’ to stable response. Transition to ‘stable response’ is defined as the week when the measure falls below the transition threshold for two consecutive weeks and (during the observation period) never returns above threshold for two or more weeks.

FIG. 5A illustrates example regions showing correlation between the transition week and the white matter microstructure damage (p<0.05), measured by both fractional anisotropy (FA) and radial diffusivity (RD) or FA and axonal diffusivity (AD), in (i) ventromedial frontal (vmF), (ii) anterior hippocampus (aHC), (iii) insular (Ins), and (iv) dorsal anterior and posterior cingulate cortex (dACC and PCC) according to aspects of the present disclosure.

FIG. 5B illustrates an example graph of post-hoc scatter plots of identified regions versus the transition to ‘stable response’ week in dACC (top) and vmF (bottom) according to aspects of the present disclosure.

FIG. 5C illustrates example significant correlation of dACC FA and functional connectivity between SCC and MCC with the number of episodes in a lifetime using all nine subjects (excluding one subject due to artifact, p<0.05) indicated by orange dot in coronal section (top) according to aspects of the present disclosure. These regions are directly connected from the stimulation target via the cingulum bundle (bottom, yellow lines) which also contains the FA and RD abnormality described in 5A.

FIG. 5D illustrates an example graph of post-hoc correlation between FA and functional connectivity indicates a significant relationship between FA in the dACC and functional connectivity of SCC and MCC (p<0.05).

FIG. 5E illustrates an example graph of a correlation between number of episodes in lifetime and functional connectivity of SCC and MCC and FA (p<0.05) according to aspects of the present disclosure.

FIG. 6A illustrates an example facial expression classifier according to aspects of the present disclosure. Facial landmarks may be extracted from each frame of video of clinical interviews following which facial representation features (e.g., action units, gaze, and pose, etc.) can be estimated for each frame. Secondary features including first and second order moments of the estimated features in 5 minute windows may be used as features for classification. Logistic regression classifiers may be trained for each individual participant's features to classify ‘sick’ and ‘well’ states. The features from the intermediate period (Weeks 5-20) are then projected through the trained classifiers to get prediction probability which serves as a measure of behavioral state.

FIG. 6B illustrates example receiver operating characteristic (ROC) curves of logistic regression classifier in classifying ‘sick’ and ‘well’ states within individual participants according to aspects of the present disclosure. Grey lines indicate the mean ROC curve of individual participants. Black lines indicate the mean ROC curve across participants.

FIG. 6C illustrates a graph of trajectories of facial expression classifier prediction for six patients according to aspects of the present disclosure. Red line indicates the relapsed responder and grey lines indicate the other 5 typical participants. Black line indicates the average of the five typical participants. Error bars indicate standard deviation.

FIG. 6D illustrates an example graph discriminative component vs facial expression classifier prediction from weeks 5¬120 for the 5 typical responders according to aspects of the present disclosure. Dots indicate weeks for individual participants and the line indicates least-square fit regression.

FIG. 6E illustrate an example graph of a correlation between transition weeks inferred from SDCs and facial expression classifier predictions according to aspects of the present disclosure. Dots indicate individual participants. * indicates p<0.05.

FIG. 7A illustrate an example overview of facial expression classifier analysis. Facial landmarks are extracted from each frame of videos of clinical interviews and facial representation features (action units, gaze and pose) are estimated for each frame according to aspects of the present disclosure. Separate logistic regression classifiers can be trained for each individual participant's features to classify ‘sick’ and ‘stable response’ states. The features from the intermediate period (Weeks 5-20) for each participant can then be projected through the corresponding trained classifiers to get prediction probability which serves as a measure of behavioral state.

FIG. 7B illustrate example receiver operating characteristic (ROC) curves of face classifier in classifying ‘sick’ and ‘stable response’ states within individual participants according to aspects of the present disclosure. Grey lines indicate the mean ROC curve of individual participants. Black lines indicate the mean ROC curve across participants.

FIG. 7C illustrate an example muscle heat map showing consensus changes in action unit intensities between the ‘sick’ and ‘stable response’ states across all participants according to aspects of the present disclosure. Red color indicates increases while green color indicates decreases.

FIG. 7D illustrates example graph of trajectories of face classifier output for five typical responders. Grey lines indicate individuals and the black line indicates the average according to aspects of the present disclosure. Error bars indicate standard deviation.

FIG. 7E illustrate an example graph of SDC vs face classifier output from weeks 5-20 for the 5 typical responders. Dots indicate weeks for individual participants and the line indicates least-square fit regression.

FIG. 7F illustrate an example graph of correlation between transition weeks inferred from the SDC and face classifier output. Dots indicate individual participants. *p<0.05.

FIG. 8A illustrates an example graph of a change in Hamilton Depression Rating Scale (HDRS) and SDCs before and after the week of stimulation intensity change according to aspects of the present disclosure. Grey lines indicate the change relative to the week stimulation intensity was changed for each individual change in stimulation intensity. Black lines indicate the average across all changes. Error bars indicate standard deviation. * indicates p<0.05 one-sample t-test.

FIG. 8B illustrates a graphical representation of a correlation between white matter integrity and transition to stable response. Regions showing correlation between fractional anisotropy (FA) and transition weeks are displayed in blue. Colored lines indicate the boundaries of major white matter tracts that are targeted by stimulation. vmF—ventro-medial Frontal cortex, ins—insula, sCC—sub Callosal Cingulate, vSt—ventral striatum, pCC¬posterior Cingulate Cortex, aHC—anterior Hippocampus.

FIG. 8C illustrates example scatter plots of average FA of identified regions of interest versus transition weeks according to aspects of the present disclosure. Red dots indicate individual participants. Blue line indicates the line of best fit. Gray shaded area indicates error of fit.

FIG. 9A illustrates example graph of a change in the SDC (Left) and HDRS-17 (Right) before and after the week of stimulation voltage change according to aspects of the present disclosure. Grey lines indicate the change relative to the week of a stimulation voltage change for each individual adjustment of stimulation voltage. Black lines indicate the average across all changes. Error bars indicate standard deviation. *p<0.05 (Wilcoxon signed rank test).

FIG. 9B illustrates example graph of SDC in an out-of-sample participant who was a relapsed responder according to aspects of the present disclosure. Blue line denotes HDRS-17 and red line denotes the SDC inferred from LFP features not used for training the classifier or SDC. The SDC increased above the threshold of 0.5 (grey dashed line) indicating relapse (red arrow) at week 12, indicating the relapse five weeks before it was observed in the HDRS-17 at week 17 (blue arrow). Purple arrows indicate changes in stimulation voltage levels. Note that stimulation voltage change did result in a SDC decrease as shown in panel A; however the SDC did not stabilize until 3 stimulation voltage changes were made. Notably the final voltage in this patient was comparable to the average voltage in the typical responders.

FIGS. 10A-10C illustrates an example of permutation feature importance using shuffle based technique to determine the contribution of features to classification performance according to aspects of the present disclosure. Since the features were correlated, a dendrogram based clustering may be used to identify clusters of features (distance threshold=1). Features within a cluster may be permuted jointly to generate shuffled datasets (n=100) which may then be evaluated using the classifier trained on the original dataset. The decrease in performance of the shuffled datasets provides a measure of the feature's contribution to classifier performance. FIG. 10A illustrates an example adjacency matrix based on spearman correlation between spectral features. Hotter colors indicate positive correlation. FIG. 10B illustrates an example dendrogram based clustering of features. FIG. 10C illustrate an example difference in area under ROC between classifier trained on original dataset and shuffled datasets.

FIG. 10D illustrates an example graph of shuffle-based procedure to determine significance of changes in SDC with changes in stimulation voltage according to aspects of the present disclosure. For example, in each participant, two weeks when the stimulation voltage change was not made were chosen randomly and the change in SDC one week after each of the weeks were calculated. These changes were pooled across participants and a Wilcoxon signed-rank test was run to obtain the t-value. This procedure was repeated 10000 times to obtain the null distribution denoted in blue above. The true statistic, i.e., the t-value of changes in SDC one week after stimulation voltage change, is denoted by the red line. An empirical p-value was determined from these two measures. This empirical p-value was statistically significant at the threshold of 0.05 (p=0.034).

FIG. 11A illustrates an example graph of spectral discriminative component vs relative HDRS from weeks 5-20 for the 5 typical responders according to aspects of the present disclosure. Dots indicate weeks for individual participants and the line indicates least-square fit regression.

FIG. 11B illustrates an example graph of spectral discriminative component vs relative MADRS from weeks 5-20 for the 5 typical responders according to aspects of the present disclosure. Dots indicate weeks for individual participants and the line indicates least square fit regression.

FIG. 11C illustrates an example graph of a correlation between transition weeks inferred from HDRS and SDCs according to aspects of the present disclosure. Dots indicate individual participants.

FIG. 11D illustrates an example graph a correlation between transition weeks inferred from MADRS and SDCs according to aspects of the present disclosure. Dots indicate individual participants.

FIG. 12 illustrates example graphs indicating changes in features relative to the feature strength on transition week. * indicates p<0.05 from a one sample t-test (n=5) according to aspects of the present disclosure. There was no conclusive evidence for change in any features. Features that are significantly different from the week of transition suggest changes in these features over time may be driving the changes in SDCs that are identified as transitions.

FIG. 13 illustrates example participant video frames illustrating action unit differences according to aspects of the present disclosure.

FIG. 14 illustrates an example graph of changes in features underlying SDCs around stimulation intensity increase. *indicates p<0.05, one sample t-test (n=8) according to aspects of the present disclosure. Changes in stimulation intensity resulted in significant increases in left alpha band power and right high beta band power relative to the week of stimulation change, but not in left low beta band power or right gamma band power.

FIG. 15A illustrates an example graph of the discriminative component on the week of stimulation intensity change and change in discriminative component post-stimulation intensity change according to aspects of the present disclosure.

FIG. 15B illustrates an example graph of the correlation between the week of stimulation intensity change and change in discriminative component post-stimulation intensity change according to aspects of the present disclosure.

FIG. 16A illustrates an example graph of HDRS-17 scores across different phases according to aspects of the present disclosure. Dashed lines indicate the score at which the participant is considered to be a responder (based on 50% decrease in HDRS-17). Dotted line indicates a HDRS-17 score of 8 below which participants are considered to be in remission.

FIG. 16B illustrates an example graph of MADRS scores across different phases according to aspects of the present disclosure. Dashed lines indicate the score at which the participant is considered to be a responder (based on 50% decrease in MADRS). Dotted line indicates a MADRS score of 10 below which participants are considered to be in remission.

FIG. 17 illustrates example graphs of change in power relative to last week of post-surgical period without stimulation from the beginning of the observation phase to the end of observation phase according to aspects of the present disclosure.

FIG. 18A illustrates an example graph of information flow from low-dimensional latent space components to classifier prediction according to aspects of the present disclosure.

FIG. 18B illustrates an example graph of classifier performance in leave-one-participant out cross-validation for different datasets according to aspects of the present disclosure. Reconstructed data refers to data reconstructed from GCE using all components. Performance of the classifier in datasets reconstructed by randomizing discriminative and non-discriminative components is shown in magenta and cyan bars. Randomizing the discriminative component of the held-out dataset may affect the classifier performance significantly indicating that the association between data and classifier prediction is impaired which in turn confirmed that the GCE did not overfit to the training dataset.

FIG. 18C illustrates an example graph of a receiver operating characteristic curve for neural network classifier trained on the reconstructed data to distinguish ‘sick’ vs ‘well’ state according to aspects of the present disclosure.

FIG. 19 illustrates example graphs of a change in feature strength caused by the change in latent factors according to aspects of the present disclosure.

FIG. 20A illustrates example graphs of coefficients for the different terms in the polynomial fit for each feature when the discriminative and non-discriminative components were varied according to aspects of the present disclosure.

FIG. 20B illustrates example graphs of the goodness-of-fit of the polynomial model according to aspects of the present disclosure.

FIG. 21A illustrates example graphs of a distribution of SDCs for ‘sick’ and ‘well’ states according to aspects of the present disclosure. Dotted lines indicate the threshold value chosen for the transition.

FIG. 21B illustrates example graphs of a cumulative distribution of SDCs for ‘sick’ and ‘well’ states according to aspects of the present disclosure. Dotted lines indicate the threshold value chosen for the transition and the corresponding proportion of ‘well’ state data.

FIGS. 22A and 22B illustrate example graphs of facial expression state and clinical assessments according to aspects of the present disclosure.

FIG. 23A illustrates example trajectories of relative HDRS and discriminative component for illustrates example trajectories according to aspects of the present disclosure. DBS905 is relapsed responder.

FIG. 23B illustrates example trajectories of relative facial expression classifier prediction and discriminative component for illustrates example trajectories according to aspects of the present disclosure. DBS905 is relapsed responder.

FIG. 24 illustrates example trajectories of relative HDRS-17 and spectral discriminative component for individual participants. P001 is the relapsed respondercording to aspects of the present disclosure. Vertical dashed line indicates the week when stimulation voltage was increased.

FIG. 25 illustrates example graphs indicating how well the SDC sick/stable response designation matches the HDRS-defined sick/stable response state in typical responders according to aspects of the present disclosure. The blue line denotes the HDRS-state while the red line denotes the SDC-state using a threshold of 0.5.

FIG. 26 illustrates example trajectories of face classifier output and spectral discriminative component for individual participants according to aspects of the present disclosure. P001 is the relapsed responder. Vertical dotted line indicates the week when stimulation voltage was increased.

FIG. 27A illustrates an example graph of HDRS-17 and SDC over time according to aspects of the present disclosure. P002 SDC (red line) indicated stable response, defined using the criteria in FIG. 4E, at Week 13 while HDRS-17 (blue line) indicated ‘stable response’ at Week 20. Stimulation voltage change (Purple arrows) did not decrease HDRS-17 but decreased SDC.

FIG. 27B illustrates an example graph of psychic anxiety item (orange) of HDRS-17 and depressed mood (blue) over time according to aspects of the present disclosure. Psychic anxiety item (orange) of HDRS-17 increased contribution to total HDRS-17 while depressed mood (blue) remained constant suggesting the elevated HDRS-17 beyond week 13 (when SDC indicated stable response) may have been sustained by increase in anxiety. Clinical notes support this hypothesis—‘Biggest treatment issue is an internal resistance to the loss of depression and fears about what that means for her, including confronting feelings of loneliness and emptiness’.

FIG. 28 illustrates example graphs of the psychomotor retardation subcomponent (blue lines) of HDRS-17 along with changes in beta band activity on the left and right hemispheres according to aspects of the present disclosure. Changes compared in beta band activity (magenta lines) with the retardation subscore of the HDRS-17 that captures motor changes observed in patients including slowness of thought and speech, impaired ability to concentrate, decreased motor activity. There is no clear relationship between beta band activity and retardation across the 6 participants. Only P001 appears to have a negative correlation between beta band activity and retardation, but a similar trend was observed between beta band activity and all items in HDRS-17.

FIG. 29 illustrates example graphs of trajectories of participants excluded from analysis.

### DETAILED DESCRIPTION

Unless otherwise defined, all technical and scientific terms used herein have the same meaning as commonly understood by one of ordinary skill in the art to which the methods described herein belong.

The singular form “a”, “an” and “the” include plural referents unless the context clearly dictates otherwise. These articles refer to one or to more than one (i.e., to at least one). The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

The term “about” as used in connection with a numerical value throughout the specification and the claims denotes an interval of accuracy, familiar and acceptable to a person skilled in the art. In general, such interval of accuracy is +/−10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

The term “exemplary” means serving as a non-limiting example, instance, or illustration. As utilized herein, the terms “e.g.,” and “for example” set off lists of one or more non-limiting aspects, examples, instances, or illustrations.

Treatment-resistant depression (TRD) patients experience a wide variety of debilitating symptoms, including persistent negative mood, anhedonia, psychomotor retardation, and suicidal thoughts. While many TRD patients receiving experimental SCC DBS have responded to continuous stimulation with durable symptom relief, the clinical management of these patients is often complex due to a number of interacting factors. In particular, the progress of antidepressant response is nonlinear and can be different for each individual, often involving periods of mood fluctuation for which there is no absolute unanimous clinical interpretation. Sufficient objective markers of depression severity may not be available causing some psychiatrists to rely on clinical intuition to decide whether to make changes to stimulation parameters or apply a watchful waiting approach. For example, clinical teams may use interviews and symptom surveys such as the Hamilton Depression Rating Scale (HDRS) to quantify depression severity, but these rater-dependent measures are often obscured by various non-specific factors such as subjective recall biases and reactions to environmental circumstances. While depression diagnostic criteria can be based on negative mood and anhedonia that are sustained over a period of weeks, patients may experience normal transient short-term mood fluctuations due to a variety of factors (e.g., stressful life events, interrupted sleep, transitory anxiety) that are reflected in the HDRS and confound the measurement of core depression symptom changes.

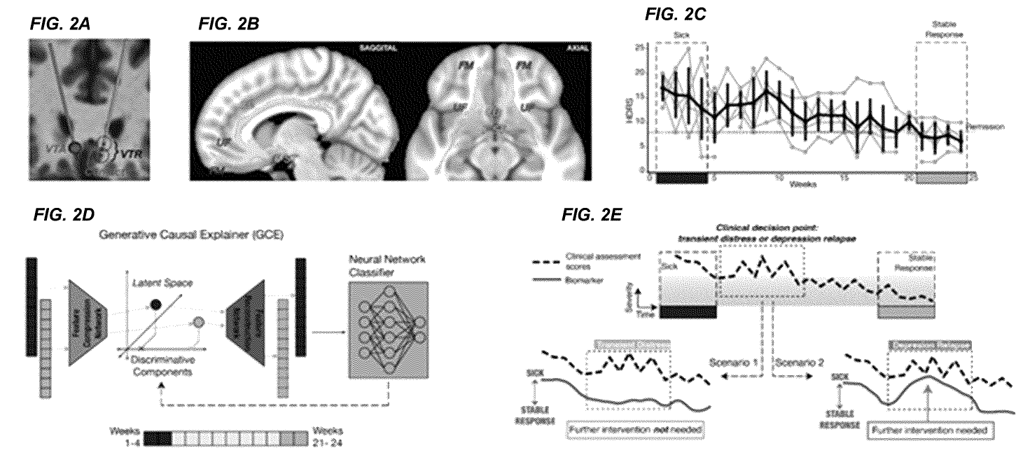

Methods and systems are described herein for a defining a data-driven brain-based biomarker of stable depression recovery that can be used to differentiate clinically acute scenarios from periods of normal transient distress (e.g., as shown in FIG. 2E). First, the high response rate combined with the heterogeneous trajectories to recovery achieved in the clinical cohort described here provides a unique opportunity to explore inter-subject differences in the path to achieving antidepressant response. While most of the current understanding of SCC electrophysiology dynamics in DBS arises from acute measurements (e.g., intraoperative/perioperative LFP), new neurotechnology platforms allowing long-term electrophysiology monitoring provided an opportunity to study longitudinal changes with DBS over a 24-week treatment period as patients achieved stable recovery. Recent XAI developments have introduced approaches to explaining ‘black-box’ machine learning models, providing a powerful novel framework for data-driven discovery of effective biomarkers.

The biomarker accurately identifies depressive and recovered states, tracks individual recovery trajectories and predicts relapses, provides evidence of differential acute and sustained neuronal network adaptations, and concords with objective changes in facial expression over the course of recovery. Furthermore, a multimodal analysis based on this brain signal shows that specific structural and functional deficits in the targeted white matter network reflect baseline disease severity (number of life-time depressive episodes) and time to respond to DBS, demonstrating individual differences that account for variable recovery trajectories with SCC DBS. Taken together, these results advance the existing practice of SCC DBS by providing actionable objective information to support personalized clinical management, provides new insight into the complex relationship between functional, structural and behavioral factors involved in patient-specific recovery, and motivates further research in using multimodal measurements to advance the treatment of depressive disorders.

Distinguishing transient normal fluctuations in mood and arousal (positive or negative) from depression symptom change (improvement, plateau, relapse) is especially important because patients often experience a period of instability as they transition between sick and stable well that is not captured by current behavioral assessments. This intermediate state contains unpredictable but transitory mood fluctuations superimposed on critical transitions that represent progression toward sustained therapeutic response. Using the biomarker will help overcome the limitations of current behavioral assessments and provide an objective measure of brain function to determine treatment strategy.

The classifier approach used for identifying facial features provides another behavioral measure that is not affected by subjective biases found in interview based behavioral assessments. This approach, in contrast to available approaches, uses a personalized approach to track changes in features that capture the difference between discrete ‘sick’ and ‘well’ states.

Deep brain stimulation (DBS) of the subcallosal cingulate cortex (SCC) has been effective in treating patients with treatment-resistant depression, but it is unclear how chronic DBS alters neural activity to produce stable therapeutic response. Understanding the chronic neural activity changes is crucial for improving the therapy in the context of variability in individual patient recovery trajectories and increasing interest in adaptive neurotechnologies for closed-loop stimulation. Here the inventors collected local field potentials (LFP) from six participants undergoing DBS of a tractography-defined white matter target in SCC, with each patient showing variable symptom trajectories preceding robust therapeutic response at the 24 week endpoint. Using a novel machine learning approach, the inventors identify changes in SCC LFP dynamics that reflect differences in clinically defined sick/well states, that differ from acute stimulation effects previously reported, and that respond to dosage changes. The inventors demonstrate that this biomarker tracks individual variation in stable recovery identified through data-driven analysis of facial expressions, which the inventors also show is predicted through anatomical anomalies within the targeted white matter network. Our results suggest that adaptation of the local network driven by chronic DBS may contribute to long-term symptom recovery and demonstrate functional biomarkers of stable recovery that may inform future clinical trials or closed-loop neuromodulation systems.

In this work, the inventors identify long-term changes in SCC dynamics that accompany stable depression recovery with DBS. The present disclosure relates to a method and system for deriving biomarkers from electrophysiological measurements to track major depressive disorder (MDD) disease state and identify critical transitions between discrete disease states. Also disclosed are methods for utilizing facial features to track changes in facial feature between discrete disease states and a structural connectivity brain map that predicts when the transitions may happen in a given patient. In some aspects of the disclosure, the biomarkers of disease state could be used to (1) make on-line changes to Deep brain stimulation (DBS) stimulation parameters (i.e., known as “closed-loop” DBS stimulation) or (2) optimally time the introduction of needed adjunctive treatments (e.g., behavioral activation, cognitive therapy, medication dose reductions, etc.).

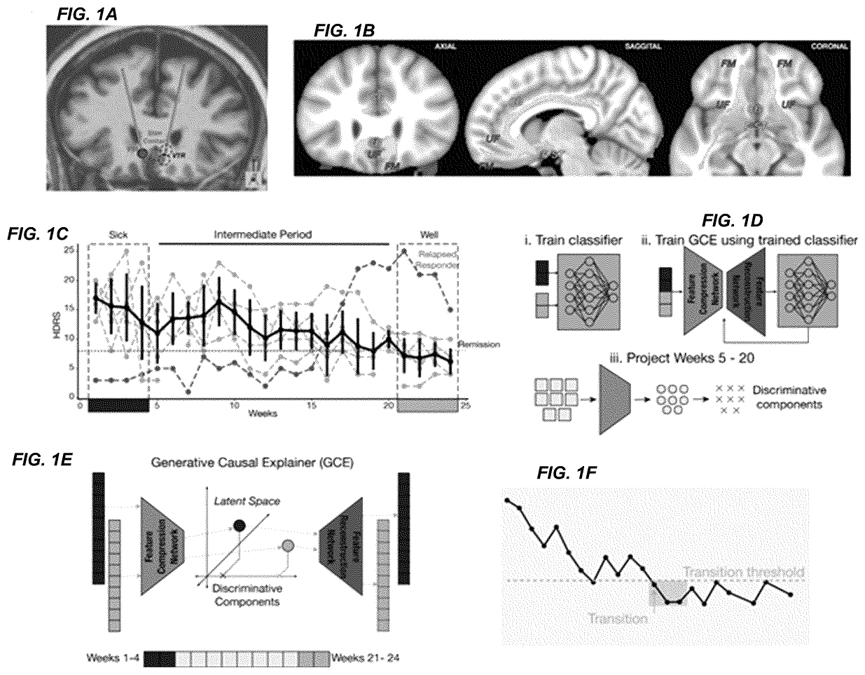

Specifically, the inventors recruited participants with TRD who underwent DBS of an SCC white matter target (FIG. 1A) using an implanted pulse generator that also served as a long-term LFP acquisition system. The target was personalized for each individual participant based on diffusion tensor imaging and LFP was collected over a period of 24 weeks during treatment (see Methods). The treatment was highly effective for the cohort, yielding a response rate of 90%. However, there was variability in the individual response trajectories over time (FIG. 1C), with patients experiencing a period of idiosyncratic variability in symptoms before reaching stable recovery (e.g., persistent symptom remission) at different times. This provided a unique opportunity to identify changes in dynamics that can explain the symptom recovery over the 24 weeks as well as the differences in response trajectories. The inventors used a neural network classifier on the cohort to verify that there are common SCC dynamics from the first 4 weeks (termed as ‘sick’ state) and the last 4 weeks (termed as ‘well’ state) that are indeed distinct. The inventors then used a novel XAI approach called the generative causal explainer (GCE) and derived ‘spectral discriminative components’ (SDCs).

SDCs are low-dimensional latent representations of the features in the SCC dynamics that are being used by the classifier. SDCs identify the features that exhibit long-term change and serve as a biomarker of symptom recovery. To verify if the changes in SDCs that the inventors observed are relevant for DBS mediated recovery, the inventors inferred SDCs in the intervening period between the beginning and the end of the therapeutic period and compared it to two different behavioral measures of depression: scores from clinical assessments and data-derived markers of recovery from facial expressions.

The current standard for assessing symptom severity in DBS trials is clinician-administered survey-based assessments such as the Hamilton Depression Rating Scale (HDRS), which are confounded by various factors including retrospective recall bias, mood fluctuations unrelated to depressive episodes and poor longitudinal invariance. Facial expression analysis, which forms an important part of affective computing research in identifying depression and depression severity from audiovisual features, overcomes some of the limitations of clinical assessments and may complement these standard assessments in getting a clearer indicator of the patient's depressive state. These approaches focus on action units characterized by stereotypical movements of eyes and lips as well as head movements such as pose and gaze. While considerable progress has been made in detecting depression from facial features, there have been very few tools for tracking depressive symptoms over time using facial features. Therefore, the inventors developed a personalized approach to tracking facial expressions for each participant over the treatment period. Following the same rationale as the LFP analysis, the inventors used classifiers to distinguish facial features from the ‘sick’ and ‘well’ states. The inventors then used the personalized classifiers to predict the participant's facial expression state in the intermediate period and compared the facial expression states to SDCs. In addition, the inventors identified the week at which the patient's trajectory transitions from the ‘sick’ state to a stable ‘well’ state. Patients undergoing SCC DBS for TRD exhibit a characteristic response trajectory with a relatively smooth initial decrease in symptoms followed by a period of emotional reactivity and uneven symptom changes over time, followed by a stabilization of the decreased symptoms. The inventors inferred when the recovery stabilizes based on SDCs, clinical assessments, and facial expression states, and verified concordance.

The inventors found that a common set of multivariate features of SCC dynamics could distinguish ‘sick’ and ‘well’ states in all the participants. The features include increases in low beta band (13-20 Hz) power in the left hemisphere and gamma band (30-40 Hz) power in the right hemisphere. While the initial changes after therapeutic stimulation onset match the acute stimulation effects reported previously, the long-term changes were marked by an opposite effect. Importantly, SDCs that captured the changes in these features tracked recovery from symptoms as measured using the facial expression state and responded to changes in stimulation intensity made during the course of treatment. Further, the time to stable recovery inferred from the SCC dynamics was correlated with white matter irregularities in the main tracts that form the target of stimulation. The results suggest that the long-term changes in SCC dynamics associated with recovery of symptoms with DBS may be mediated by an adaptive process different from the effects of acute stimulation.

The inventors extracted spectral features from local field potentials for the classification of the first 4 weeks and the last 4 weeks of the 24 week treatment period. Five out of the six participants started the treatment phase with elevated depressive symptoms (HDRS Weeks 1-4: 15.2±0.83, mean±std) and ended the treatment phase with significantly reduced depressive symptoms (HDRS Weeks 21-14: 6.9±2.39; paired t-test, t=6.12, p=0.003). The five participants reached clinical response criteria based on HDRS (greater than 50% decrease in score) and four out of the five participants achieved remission (a HDRS score less than 8). Based on the relative stability of HDRS at the beginning and end of the treatment phase, the participants were considered to be in a ‘sick’ state during the first four weeks and in a ‘well’ state the last four weeks of this period. One participant (DBS905) exhibited an atypical pattern in which their HDRS began the treatment phase low but worsened towards the end (FIG. 1B) and was considered a non-responder at the registered endpoint of the treatment phase. The inventors termed this participant ‘relapsed responder’.

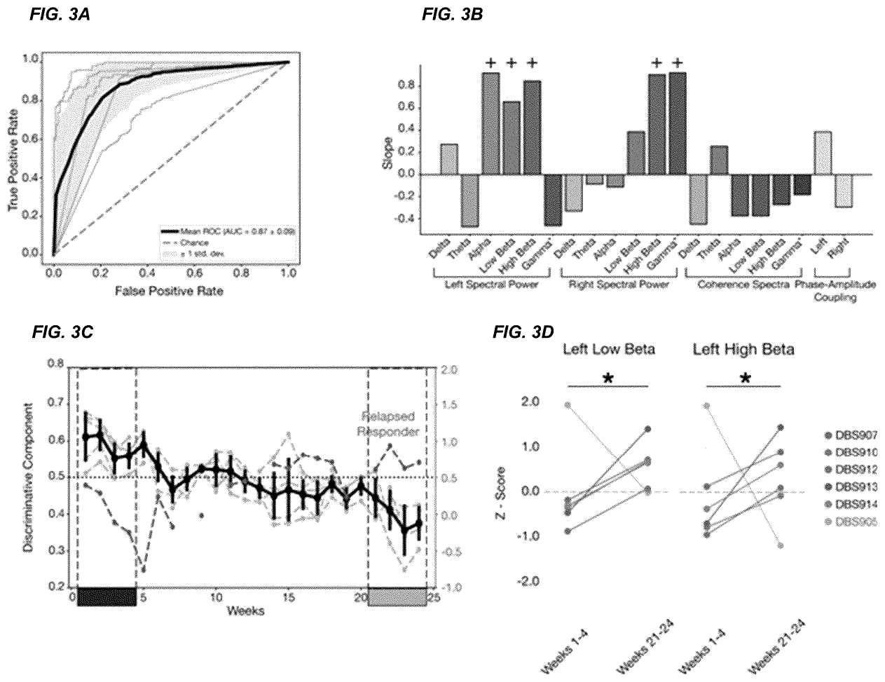

A neural network classifier (with leave-one-participant-out cross-validation) was able to distinguish the ‘sick’ and ‘well’ states (AUROC: 0.87±0.09; FIG. 3A) in the 5 responders, suggesting recovery from depression is reflected in similar electrophysiological changes across participants. As the relapsed responder exhibited a different trajectory, the inventors did not include the participant's LFP data in the classifier analysis but used it as a validation set for GCE analysis.

The inventors trained a generative causal explainer (GCE) to identify spectral discriminative components (SDCs), which are low-dimensional latent representations of the spectral features that capture the difference between the sick and well states as determined by the neural network. Thus, in our case, SDCs serve as markers of LFP state, with higher values indicating ‘sick’ state and lower values indicating ‘well’ state.

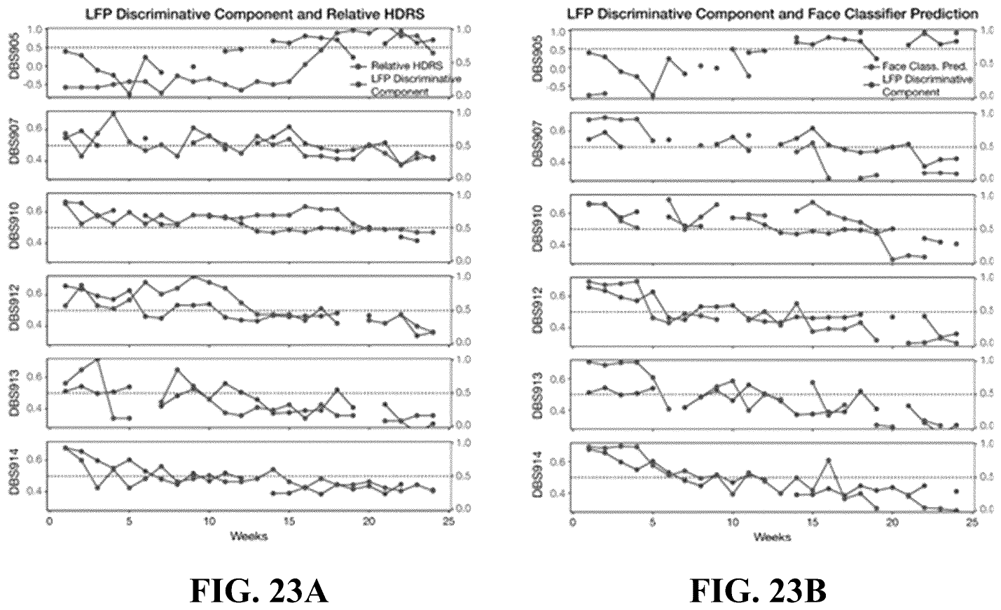

Following this the inventors inferred the SDCs for the intermediate period (weeks 5-20) to estimate the trajectory of LFP changes from the ‘sick’ state to the ‘well’ state (FIG. 3C). Interestingly, the SDCs inferred for the atypical responder followed an overall trend that was broadly similar to their HDRS (low at the beginning of the treatment phase and high at the end), with an opposite trajectory from the typical responders (FIG. 23A, top row). As neither the classifier nor the GCE were trained with the data from the relapsed responder, the fact that the inferred SDC followed the general trend of symptoms suggests that the SDCs capture LFP state underlying depressive symptoms.

The inventors identified the features underlying SDCs by leveraging the generative property of GCE. By projecting variations in SDCs through the feature reconstruction network, the inventors were able to identify features that exhibited the most changes. The inventors fit a 2nd order polynomial model to characterize how changes in SDCs affected changes in features. The slope of the changes in features when SDCs were varied is shown in FIG. 3C. A positive slope indicates an increase in the feature's magnitude when SDCs changed from the ‘sick’ state to ‘well’ state while a negative slope indicates a decrease in the feature's magnitude.

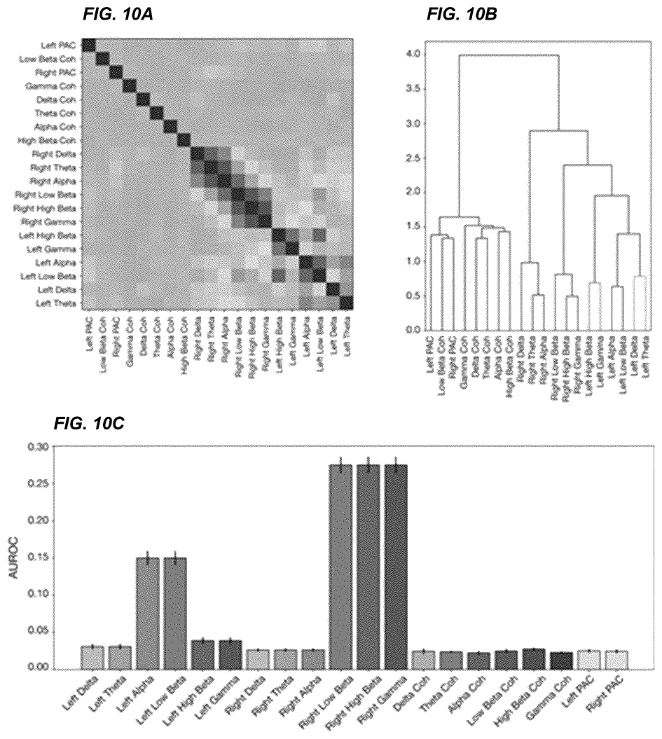

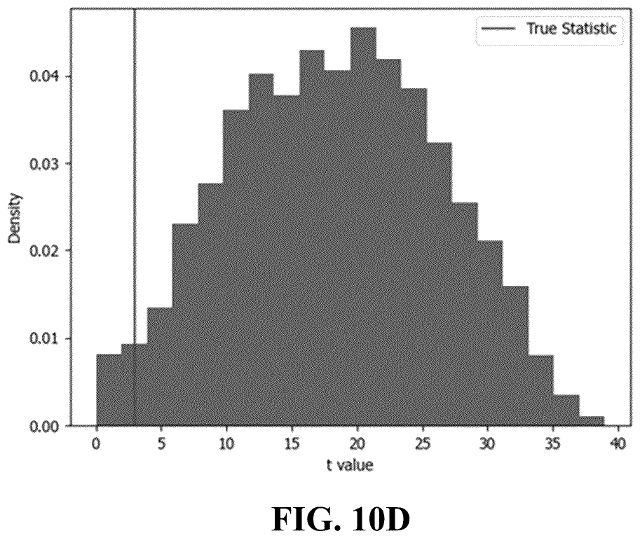

Changes in SDCs resulted in changes in many spectral features with the largest changes observed in left alpha (8-12 Hz), left low beta (12-20 Hz), left high beta (20-30 Hz), right high beta, and right gamma band power (30-40 Hz). All of these features exhibited an increase suggesting the difference between ‘sick’ and ‘well’ states is driven by bilateral increase in beta/gamma power in SCC. A similar subset of features was identified to be important for classification using a clustering-based permutation feature importance method (FIGS. 10A-10C).

The identified features (especially beta band power) have been previously reported to respond to stimulation in acute stimulation experiments. Acute intraoperative stimulation of SCC has been shown to decrease beta band power in contrast to our results which indicate chronic stimulation results in an increase in beta band power. To compare directly against these previous studies, the inventors computed the beta band power across the treatment phase relative to the last week of the post-surgery phase when stimulation was turned OFF. The inventors found that left low beta band power was lower than the post-surgery phase in the four-week period after stimulation was turned ON (one sample t-test, t=−3.626, p=0.022) but was higher than post-surgery phase in the last 4 weeks of treatment phase (t=3.297, p=0.03) (FIG. 3C) in the five typical responders. The difference was statistically significant (paired t test with holm correction for multiple comparison, t=−6.127, p=0.002). A similar trend was observed in the left high beta band power as well (t=−4.295, p=0.01) although the increase observed in the last 4 weeks was not statistically significant (t=2.14, p-value=0.099). This indicates while the early effect of stimulation is in line with the acute effect observed in previous studies, the long-term effect is different from the early effect. In the case of the relapsed responder, the inventors observed an opposite trend suggesting the beta band power is a robust marker of depression severity.

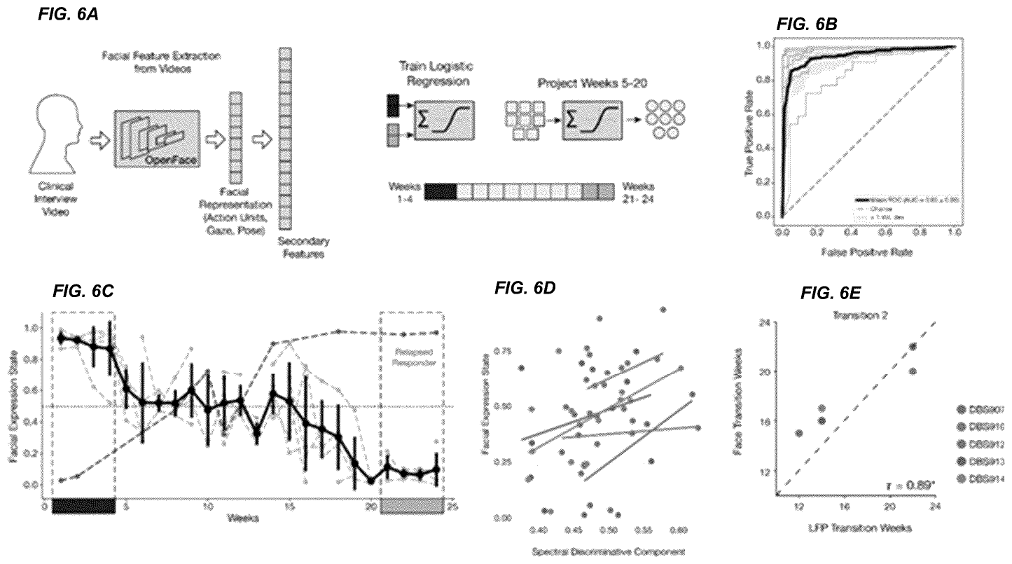

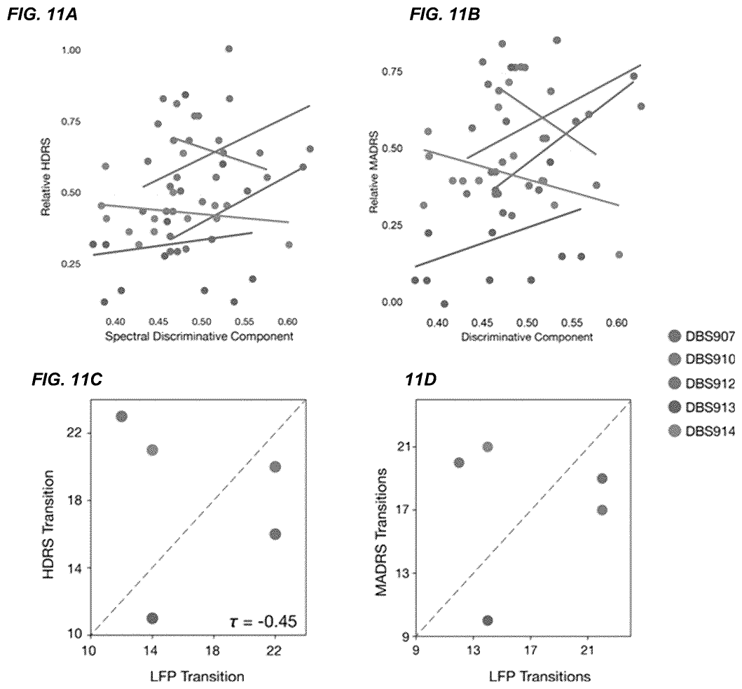

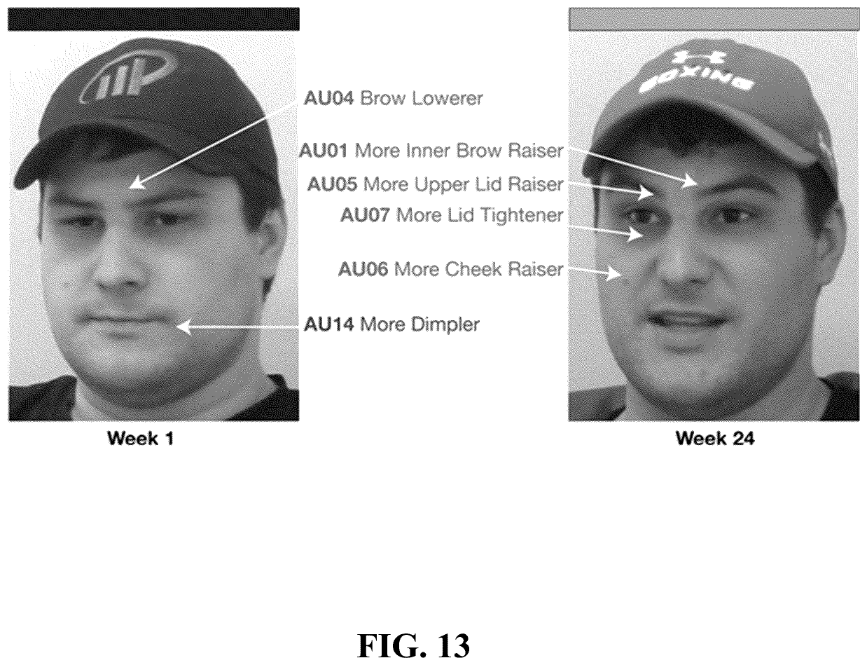

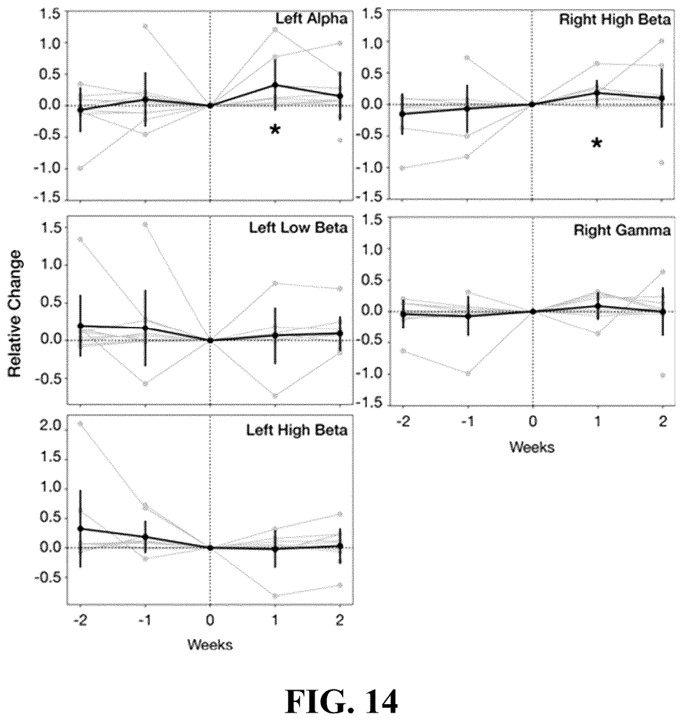

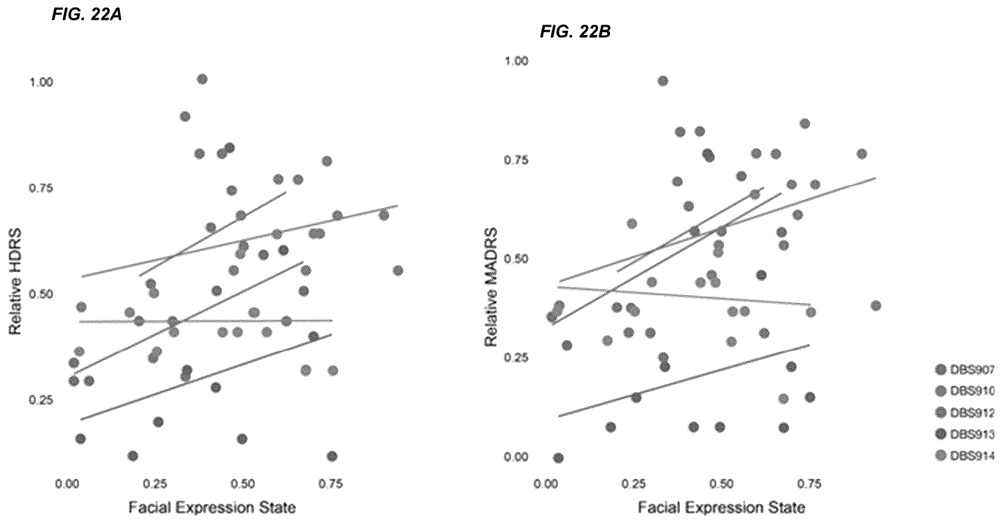

While the overall trend of the trajectories of the SDCs followed relative HDRS (FIG. 3C, FIG. 23A), the week-to-week changes in the intermediate period (weeks 5-20) did not show any significant correspondence (FIG. 11A; Linear mixed model, F(1,50.99)=1.40, p=0.24). A similar absence of relationship was observed with relative MADRS as well (FIG. 11B; Linear mixed model, F(1,50.89)=1.22, p=0.27). Given the established limitations of these clinical measures, the inventors computed an alternative behavioral measure from extracted facial expression features in videos of clinical interviews (FIG. 6A). The features comprised summary measures of facial action units eye gaze and head pose. Similar to LFP, the inventors aimed to identify differences between the ‘sick’ and ‘well’ states. However, since there are considerable inter-individual differences in facial expressions and how these features may change over depression recovery, the inventors used an individualized classifier in contrast to the LFP classifier derived for the whole cohort. Logistic regression classifiers were able to classify ‘sick’ and ‘well’ states in each individual participant separately (AUROC 0.95±0.05), suggesting that there are individualized yet consistent differences between the ‘sick’ and ‘well’ states (FIG. 6B). While the inventors found a common set of features (action units 1 and 7 and pose) across all participants, many features that distinguished ‘sick’ and ‘well’ states were unique to each participant (FIG. 13). An example of the differences in the most salient action units is displayed in FIG. 13. The inventors used these individual classifiers on facial expression features from the intermediate period to obtain facial expression state. The inventors found that the facial expression state exhibited a statistically significant relationship to clinical measures (FIG. 22). The trajectories of these facial expression states were similar to the corresponding participant's trajectories of SDCs (FIG. 6D; FIG. 23B). The inventors found a significant relationship between the facial expression classifier predictions and SDCs (FIG. 6E; Linear mixed model, F(1, 51.74)=6.54, p=0.01).

Participants undergoing DBS of SCC for TRD have been observed to undergo transitions between distinct behavioral phases over the course of the treatment phase. The inventors hypothesized that such transitions should be observable and concordant in both behavioral changes as well as brain state changes. The inventors used a threshold-based analysis on the time course of SDCs, clinical assessments, and facial expression states during the treatment phase (FIG. 1) to estimate transitions in behavioral changes and brain states respectively. The inventors did not find concordance (measured using rank correlation) between the transition weeks for transitions inferred from SDCs and HDRS (FIG. 11C; Kendall's tau=−0.45, p=0.3) or MADRS (FIG. 11D; Kendall's tau=−0.22, p=0.6). However, transition weeks inferred from SDCs and facial expression states were concordant (FIG. 6F; Kendall's tau=0.89, p=0.04). Taken together, these results suggest that the LFP changes may be associated with changes in facial expressions accompanying recovery from depression.

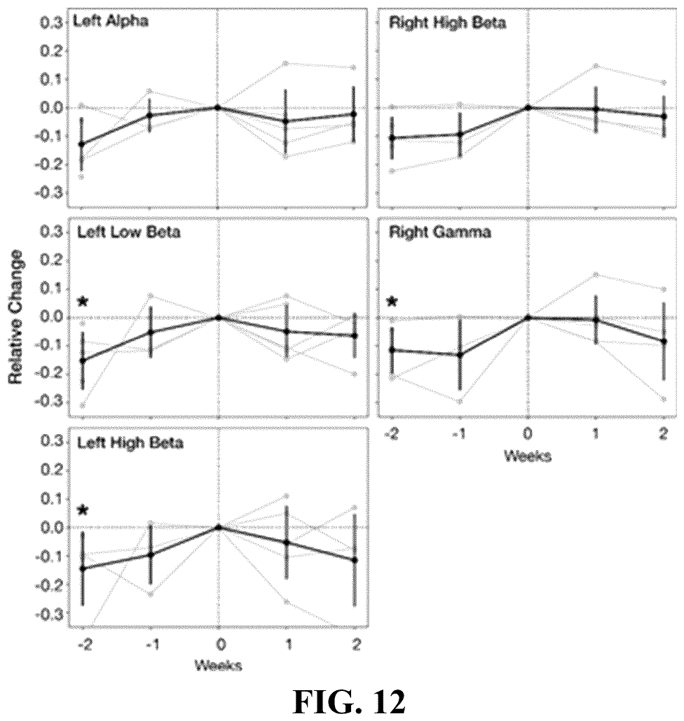

Analysis of individual features revealed that transitions in SDCs were driven mainly by changes in left low beta band power and right gamma band power (FIG. 12).

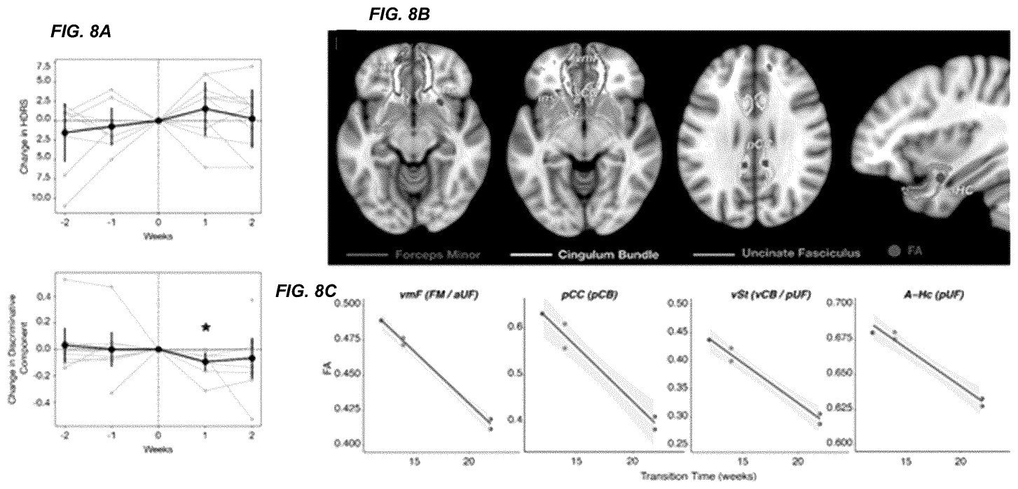

While participants typically start at the same stimulation intensity setting (3.5 V) during the course of the treatment phase, the voltage settings are changed as deemed necessary by the clinical team (Table 1). The weeks in which these changes were made varied across participants (range: 4 to 22 weeks after the beginning of therapeutic stimulation). This provides an opportunity to verify if the changes observed in SDCs are due to DBS. The inventors found that changes in stimulation intensity resulted in a decrease in SDCs (FIG. 8A, Top; ¬10.094±0.022, 1 sample t-test t=−2.6; p=0.04), suggesting that LFP features are indeed affected by stimulation intensity change. In contrast, the changes in voltage settings did not result in a significant change in HDRS scores (FIG. 8, Bottom, 1 sample t-test t=1.02, p=0.34).

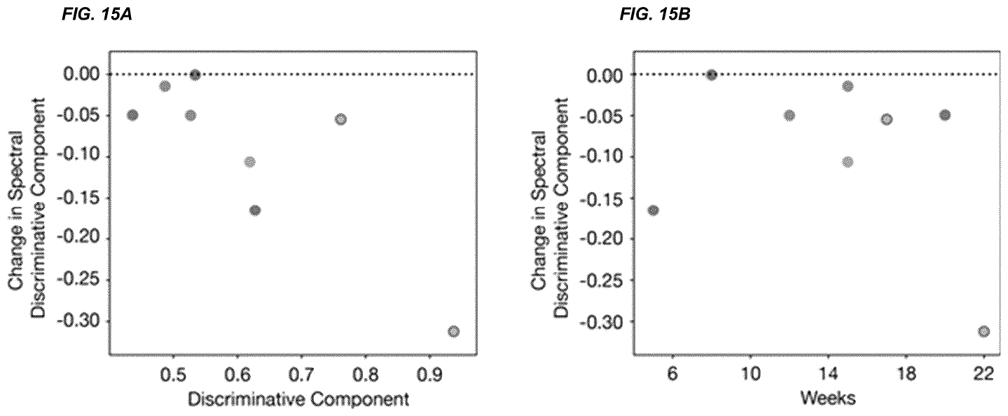

The changes in SDCs the week after stimulation intensity change depended on the value of the discriminative component the week of stimulation intensity change (FIG. 15A. Spearman rho=−0.76, p=0.02). While there was no relation between the change in discriminative component and the week on which stimulation intensity was changed (FIG. 15B. Spearman rho=−0.24, p=0.56), within responders the highest change was observed the first-time stimulation was changed with subsequent stimulation intensity changes resulting in lower changes in the SDCs.

Previous studies have shown that differences in white matter activation may lead to differences in therapeutic outcomes. The inventors hypothesized abnormalities in white matter tracts targeted by DBS may influence the transitions inferred from SDCs. The inventors found negative correlation between the transition to the stable well state and fractional anisotropy in the forceps minor and uncinate fasciculus bundles in the ventromedial frontal (vmF) cortex, the ventral striatum (vSt) and the anterior hippocampus (aHc) as well in the cingulum bundle in the posterior cingulate cortex (pCC) (FIG. 8C).

The study revealed changes in SCC LFP dynamics accompanying long-term symptom improvement in all participants in the study. The most salient changes were observed in alpha and beta, and beta and gamma band powers in the left and right hemispheres respectively. The long-term changes in these bands were generally an increase, in contrast to beta band power decreases that are typically observed as short-term changes immediately after stimulation onset. The spectral discriminative components (SDC) the inventors derived from

SCC LFP dynamics using a novel XAI method were correlated with the depressive state captured by facial expression states and responded to changes in DBS stimulation. In addition, the transition to reach a stable ‘well’ state identified from SDCs was concordant with the transition identified from facial expression states and was correlated with irregularities in the four white matter tracts targeted by DBS.

To the best of the inventor's knowledge, this is the first study to monitor long-term changes in LFP dynamics accompanying depression recovery in patients undergoing DBS. The inventors found that changes in multiple features of LFP dynamics underlie changes in SDCs, with the most dominant features being spectral power in beta and gamma bands. The initial changes observed immediately in the beta band after chronic therapeutic stimulation onset was different from changes observed towards the end of the treatment. The initial decrease in beta band power relative to the pre-treatment phase is consistent with the decrease in beta band power observed in our previous studies investigating the effects of acute intraoperative stimulation. However, the long-term changes that tracked recovery was an increase in low beta band power in the left hemisphere and high beta and gamma band power in the right hemisphere. Higher beta activity has been observed to correlate with lower symptom severity in acute intraoperative recordings. In addition, a computational model of DBS-induced recovery in the ventral cingulate cortex predicted the restoration of beta oscillations. Taken together with our observations, the increase observed in beta band power may have partly contributed to the lowering of symptoms and that recovery may be mediated by an adaptation at the local or network level.

The results indicate SCC dynamics track recovery from depression in patients undergoing DBS of SCC. In addition, changes in stimulation intensity, over the course of the treatment, induced changes in SDCs. Thus, SDCs exhibit two putative characteristics of a psychiatric biophysical signal necessary to be a candidate response biomarker for SCC DBS: correspondence to relevant behavior and engagement by the therapeutic intervention. The SDCs captured changes in the relapsed responder whose data were not used for training the machine learning models, suggesting that SDCs may be generalizable across patients undergoing SCC DBS. Thus there may be little exploration of recording sites or dynamics required in subsequent patients who undergo SCC DBS. SDCs may be used as control signals for determining when adjustments to doses are needed either in a ‘clinician-in-the-loop’ system or a fully automated closed-loop implanted DBS system. Dynamics such as beta bursts which occur at short timescales (order of seconds) are being investigated as potential control signals in DBS for Parkinson's disease with stimulation being designed to intervene at a similar timescale. In the case of depression, it is not yet clear what the optimal timescale at which intervention should be applied. Approaches that adjust stimulation parameters at the timescale of seconds have been proposed for depression. The studies are based on markers derived from sub-chronic recordings that correspond to acute changes in different aspects of mood. The results here suggest that the adaptation of brain networks to DBS needs to be taken into account both for identifying markers as well as the timescale of intervention. Thus, the timescale of intervention may be longer (order of days) in the case of depression.

The current DSM 5 criteria for major depression diagnosis requires persistence of symptoms including depressed mood, loss of interest, psychomotor disturbances, and suicidal thoughts over a period of the same 2 weeks. In particular, a depressed mood is required to be present most of the day, nearly every day. Thus, recovery from depression needs to be assessed over a long timescale (on the order of weeks). DBS targeting SCC has been demonstrated to result in sustained recovery over a period of 8 years with response rates greater than 75% and remission rates reaching 50%. Though the BROADEN study, a double-blind sham-controlled trial, did not provide conclusive evidence at the primary endpoint, a significant proportion of participants experienced response (49%) and remission (26%) with 24 months of active stimulation. Thus DBS mediated recovery from depression exhibits inter-individual variability in response trajectories. Our results presented here suggested that the difference in response trajectories is reflected in the trajectories of SDCs. In addition, our study revealed that the changes in SDCs that indicate the transition to a stable recovery were dependent on the integrity of the four white matter tracts being targeted by DBS. Thus the inter-individual variability in response trajectory may be due to variability in the engagement of networks connected by white matter tracts by DBS. In fact, the inventors have previously observed that engagement of forceps minor predicted whether patients achieved treatment response.

Beta band activity has emerged as an important marker of dysfunction across many studies investigating mood disorders. Beta band power in SCC has been shown to reflect emotional processing as well as depression severity in acute recordings. Changes in beta power in SCC induced by acute stimulation have been shown to correlate with short-term changes in symptoms. Beta band coherence between the amygdala and hippocampus was demonstrated to vary with short-term mood fluctuations. In a rodent model, beta band connectivity across multiple regions (including a homolog of SCC) was found to reflect depressive symptoms and was engaged by optogenetic stimulation. Beta band functional connectivity between subgenual cingulate cortex and posterior cingulate cortex was implicated in ruminative behavior in depression remitted patients. Interestingly, the different regions investigated in these studies form the targets of the white matter tracts being stimulated by DBS in our study. Thus beta band changes the inventors observe may reflect network-wide changes across multiple regions. Further studies incorporating electroencephalography (EEG) are necessary to capture these changes.

The inventors derived individualized facial expression states by identifying the facial expression features that exhibited the most change between the ‘sick’ and ‘well’ states. Given the small sample size, the inventors did not find a common set of features that captured the difference between the two states in all participants.

Therefore, the inventors fitted separate models for each participant which allowed us to capture the features that may be idiosyncratic to each individual. This approach limits the ability to generalize the findings across participants and requires fitting new models for each new participant. However, as data becomes available from more participants, it may be possible to identify a common set of facial expression features that reflect recovery mediated by DBS.

The presently described technology and its advantages will be better understood by reference to the following examples. These examples are provided to describe specific implementations of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### EXAMPLES

First Methods

Participants and Clinical Assessments

Ten subjects with treatment-resistant major depressive disorder were consecutively enrolled in an experimental trial using a prototype deep brain stimulation device that allowed collection of local field potentials from the stimulation site (ClinicalTrials.gov Identifier NCT01984710). Clinical findings along with inclusion and exclusion criteria have been described in. All patients provided written informed consent to participate in the study. The protocol was approved by the Emory University Institutional Review Board and the US Food and Drug Administration under a physician-sponsored Investigational Device Exemption (IDE G130107) and is monitored by the Emory University Department of Psychiatry and Behavioral Sciences Data and Safety Monitoring Board. Clinical symptom severity was assessed by an independent rater using the Hamilton Depression Rating Scale (HDRS),

Montgomery-Asburg Depression Rating Scale (MADRS), and self-reported Beck Depression Inventory (BDI) during weekly visits to the laboratory among other behavioral scales. Patients also met weekly with the study psychiatrist who adjusted stimulation current based on a combination of HDRS changes relative to the previous week and their clinical examination and interview, which included assessment of ongoing life events. Following established criteria, a decrease in HDRS scores greater than 50% of the pre-surgical average was set as the threshold for ‘response’. HDRS score of 8 was set as the threshold for ‘remission’ while a MADRS score of 10 was set as the threshold for ‘remission’. Relative HDRS and relative MADRS were computed as proportions of the pre-surgical average of HDRS and MADRS respectively.

The inventors report analysis of local field potentials (LFPs) from 6 participants listed in Table 1 during a period of 6 months from the initiation of DBS therapy. Four participants were excluded from analysis as weekly LFPs were not acquired from two participants and LFP recordings from one participant were corrupted by a gain-compression artifact and LFP recordings from another participant was corrupted by heart-beat artifacts.

TABLE 1
Participant Demographics and Clinical History

| | Age at time | | Age at | Stimulation | Number of |
|---|---|---|---|---|---|
| Participant | of surgery | | onset | voltage at | stimulation |
| ID | (years) | Sex | (years) | end of study | intensity |
| DBS905 | 67 | F | 36 | 4.5 V | 3 |
| DBS907 | 58 | F | 56 | 4.0 V | 1 |
| DBS910 | 56 | F | 46 | 5.0 V | 3 |
| DBS912 | 38 | M | 32 | 4.5 V | 2 |
| DBS913 | 44 | F | 16 | 3.5 V | 0 |
| DBS914 | | M | 26 | 4.5 V | 2 |

Subcallosal Cingulate Cortex White Matter (SCCwm) Deep Brain Stimulation (DBS)

Participants were implanted with two model 3387 electrode array leads, one in each SCC as determined from tractography. Electrodes were implanted to target the intersection of four major white matter tracts—forceps minor, cingulum bundle, uncinate fasciculus, and frontostriatal fibers (FIG. 1B). Stimulation was delivered using a voltage-controlled pulse generator (e.g., using Activa PC, or the like) which also served as the local field potential acquisition system. DBS therapy started at least 30 days after the implantation surgery to allow for recovery from surgery. Therapy consisted of bilateral monopolar stimulation on a single contact per hemisphere at 130 Hz with 90 μs pulse width. Stimulation amplitude was initially set at 3.5 V for all participants except DBS905. The initial amplitude for DBS905 was set at 3V as the participant's symptoms were below the remission threshold at the beginning of the treatment phase. During the treatment phase, location, pulse width, and stimulation frequency remained unchanged. Stimulation current was incrementally increased in steps of 0.5 V at unspecified intervals based on the study clinician's (PRP/AC) assessment of patient progress as described above. The stimulation voltage level at the end of the 6-month study period and the number of times stimulation intensity was changed in each participant are listed in Table 1.

Local Field Potential (LFP) Recordings and Extraction of Spectral Features

Local field potentials were acquired at a sampling rate of 422 Hz (e.g., using a Activa PC+S system, or the like) as differential recording from electrode contacts on either side of the stimulation contact to allow for common-mode rejection of noise as well as stimulation-related artifacts. LFPs were acquired weekly during the treatment phase in a single 15-minute session in the laboratory. Each session consisted of two segments of approximately 7.5 minutes each—one with stimulation turned ON and the other with stimulation turned OFF. Only the segments with stimulation turned OFF were included in the analysis as the presence of stimulation-related artifacts precluded functional connectivity and cross-frequency coupling analyses.

All LFP analyses were performed using custom-written scripts in Python and Matlab. LFP recorded within a session was divided into 10-second segments from which spectral power, coherence and phase-amplitude coupling were estimated. Spectral power and magnitude-squared coherence were estimated using a fast Fourier transform approach with an adaptive procedure for setting the weights of tapers. Spectral power and coherence in canonical frequency bands (Delta: 1-4 Hz, Theta: 4-8 Hz, Alpha: 8-13 Hz, Low Beta: 13-20 Hz, High Beta: 20 30 Hz, Gamma: 30-40 Hz) were then extracted as features for classification. In some instances, the upper limit of the gamma band was restricted to 40 Hz instead of 50 Hz due to the presence of device-related artifacts in the range of 40-50 Hz.

Phase-amplitude coupling (PAC) was estimated using a software library such as, but not limited to, the PACtools python library. The algorithm was used to compute the coupling between low frequency (1-15 Hz) phase and high frequency (15-45 Hz) amplitude. Comodulograms were visually inspected to identify PAC regions of interest and PAC values between delta band (1.5-3 Hz) and high-beta/gamma band (2035 Hz) were extracted as features. This procedure was followed to restrict the dimensionality of the features for the classifier, as including all the possible interactions would have considerably increased the feature set size. Thus, the overall dimensionality of the feature set was 20 (6 spectral features per hemisphere, 6 coherence features, 1 PAC feature per hemisphere).

Classification of LFP Features and Inferring Spectral Discriminative Components (SDCs)

Neural network models were used to classify LFP features using, for example, PyTorch. The parameters for the neural network models are listed in Table 2. LFP spectral features were individually scaled between 0 and 1 as a pre-processing step. A 5-fold leave-one-out cross-validation was performed at the subject level to ensure generalizability. Models were fit using LFP features from 4 out of 5 participants and tested with the features from the 5th participant and this procedure was repeated until all 5 participants served as a test case.

TABLE 2
Parameters of Neural Network Classifier

| | |
|---|---|
| Architecture | 20 × 64 × 1 |
| Activation function | Rectified Linear (ReLU) |
| Loss function | Binary cross-entropy |
| Learning rate | 0.001 |
| Optimizer | Adam |

The inventors use the generative causal explanation (GCE) framework to identify interpretable features in the data that are determinative of the classifier's output. Conceptually, GCE can be thought of as a form of dimensionality reduction in which only a subset of the low-dimensional representation has a causal impact on the classifier output (see FIG. 1F). This partitioning of the low-dimensional representation into classifier-relevant and classifier-irrelevant dimensions is accomplished by augmenting the objective of an autoencoder with a mutual information term that encourages a portion of the low-dimensional representation to influence the classifier output. The inventors call the subset of dimensions in the low-dimensional representation that are relevant to the classifier's output the “discriminative components,” and the subset of the dimensions that contribute to representing the data but do not affect the classifier's output the “non-discriminative components.”

In the present work, the GCE was implemented using two separate networks—a feature compression network that maps the data from the feature space to the low-dimensional latent space and a feature reconstruction network that reconstructs the feature space data from the latent components (FIG. 1E).

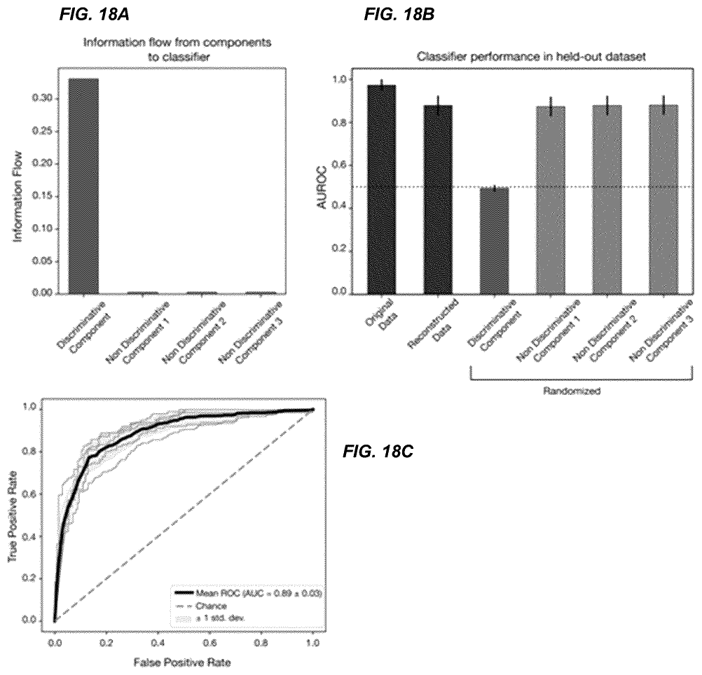

The latent components were termed spectral discriminative components (SDCs) and spectral non-discriminative components (SNDCs). The networks were trained to maximize the similarity of the reconstructed data and the true data using a loss function commonly used in variational auto-encoders as well as the information flow from the SDCs to classifier output using a loss function. The GCE was trained with features extracted from LFP collected during the first month and last month of therapy in all participants and a classifier trained on the same data. Information flow from discriminative components to classifier output was higher than that of non-discriminative components, ensuring that the SDC captures the features that determine the classifier output (FIG. 18A). A leave-one-out cross-validation was performed to make sure the model did not overfit (FIG. 18B). The reconstruction performance was evaluated by i) verifying that classification performance of a neural network classifier trained on the reconstructed data matched the performance of the classifier trained on the original data and ii) training a separate neural network classifier with original data and testing on the reconstructed data (FIG. 18C). The parameters of the networks are listed in Table 3.

TABLE 3
Parameters of GCE

| | |
|---|---|
| Feature | Feature |
| Compression | Reconstruction |
| Network | Network |

| | |
|---|---|
| Architecture | 20 × 512 × 5 |
| Activation function | ReLU |
| Learning rate | 0.0005 |
| Discriminative dimension | 1 |
| Non-Discriminative dimension | 3 |

The trained feature compression network was used to infer discriminative components of the LFP collected during months 2-5. LFP spectral features, computed in 10-second segments, were min-max scaled to the training set (LFP features from months 1 and 6) and projected through the feature compression network to infer discriminative and non-discriminative components. The SDCs were then averaged across the 10-second segments within a week.

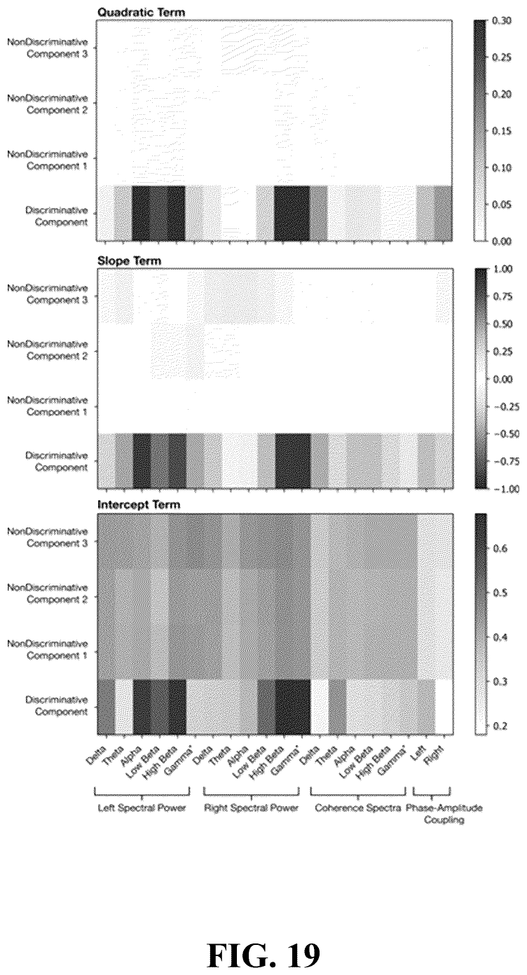

To map what features correspond to the SDC and SNDCs, the component values were varied in the latent-space and projected through the feature reconstruction network. The resulting changes in the features were fit with second-order polynomials and the magnitude of the coefficients served as an indicator of feature change between weeks 1-4 and weeks 21-24. (FIG. 19)

Identifying Facial Expression Correlates of Behavioral Change and Decoding Facial Expression State

In addition to clinical assessments, behavioral changes were estimated from facial expressions extracted from videos of participants collected during the weekly psychiatric clinical interviews where dose changes were determined. Videos were recorded using a static, tripod-mounted video camera recording at 30 frames per second. The sessions were approximately thirty minutes long.

Videos were partitioned into 5-minute windows for feature generation with the remainders discarded. Each window was processed with the Openface facial behavior analysis toolkit V2.0. This open-source software produces presence, intensity, and confidence estimations for 18 facial action units, eye gaze, and head pose vectors, as well as 68 facial landmark positions for each frame. The 30 Hz frame rate was sufficiently granular to yield a temporal resolution to capture micro expressions (<0.5-second duration) as well as macro expressions (0.5 to 4 seconds). Data from frames with less than 93% confidence was discarded. The most common reason for discarding frames was the obstruction of the subjects' faces by their hands. From these first-order features, the inventors generated second-order features consisting of envelope metrics (mean, median, quantiles, skew, kurtosis, variance) and covariance between features. From gaze and pose vectors the inventors generated velocity, acceleration, jerk, and their envelope metrics. This processing was implemented in python resulting in 1073 features overall.

Using the same rationale as for the LFP classification, the facial expression features that were most differentially expressed between the ‘sick’ (weeks 1-4) and ‘well’ (weeks 21-24) states were identified using a paired t-test and used as input to train binary classifiers for each subject. For unbalanced sample sets due to sparse recordings, SMOTE was used to oversample the minority class. A logistic regression classifier with 10-fold cross-validation was implemented in the python sklearn library to discriminate the ‘sick’ from ‘well’ state for each subject. Following this, the trained classifiers were evaluated on the samples from the intermediate period to get the probability of being in the ‘sick’ state. The classifier predictions serve as another candidate behavioral marker to track response during ongoing DBS.

Identifying Transitions in LFP and Behavioral States

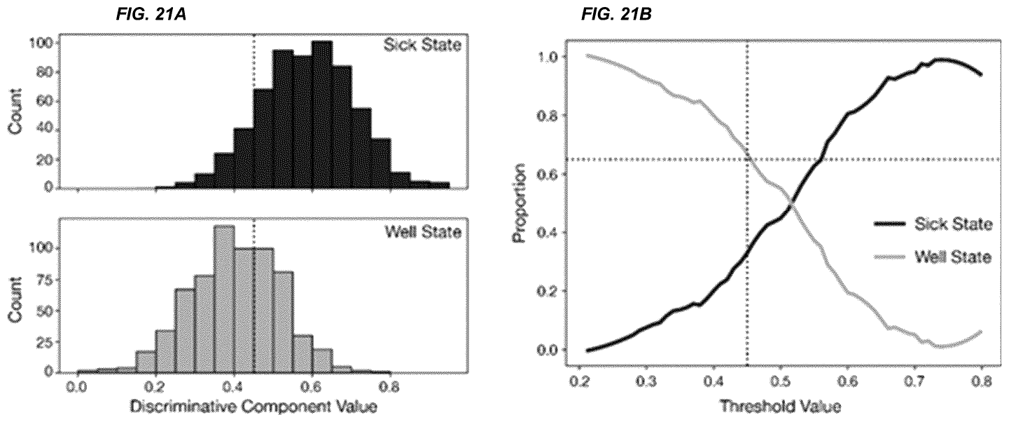

Patients receiving chronic therapeutic SCC DBS have been observed to show a characteristic response trajectory marked by a transient period of increased behavioral reactivity and instability followed by an improvement in symptoms that is sustained and stable. The inventors inferred the week at which each of the participants reached this stable response state based on weekly changes in HDRS, or facial expression classifier predictions (FIG. 1). The transition was defined as the first of three consecutive weeks when the participant's HDRS score fell below 35% of the pre-surgical average score. The SDCs follow a Gaussian distribution, with the higher end of the distribution indicating the ‘sick’ state and the lower end indicating the ‘well’ state (FIG. 21A). The cumulative distribution of the SDCs for the ‘sick’ and ‘well’ states was estimated (FIG. 12B). The threshold was determined as the value at which the proportion of ‘sick’ samples was less than 35%. (FIG. 21B). Facial expression classifier predictions provide. The second transition was defined as the week when the prediction fell below 0.35 and stayed below that threshold for 3 consecutive weeks.

Imaging Acquisition and Processing

High-resolution structural T1 and diffusion-weighted images (DWI) were acquired (e.g., using a 3T Siemens Tim Trio MRI scanner, or the like). TI-weighted image was collected using 3D magnetization-prepared rapid gradient-echo (MPRAGE) sequence with the following parameters: sagittal slice orientation; resolution=1.0 mm×1.0 mm×10 mm; repetition time (TR)=2600 ms; inversion time (TI)=900 ms; echo time (TE)=3.02 ms; flip angle=8°. DWI was acquired using single-shot spin-echo echo-planar imaging (EPI) sequence with the following parameters: 64 non-collinear directions with five non-diffusion weighted images (b0), b-value=1000 sec/mm2; number of slices=64; field of view=256×256 mm2; voxel size=2×2×2 mm3; TR=11300 ms; TE=90 ms. Additional full DWI data set with opposite phase encoding was also collected to compensate for the susceptibility-induced distortion.

All images were preprocessed using the FMRIB Software Library. T1 image was skull stripped and normalized to MNI152 template using fsl anat toolbox. DWI data underwent distortion and motion collection using the Eddy toolbox and a local tensor fitting to calculate the FA map. Tract-Based Spatial Statistics (TBSS) processing was performed for the group analysis. Briefly, individual FA images were aligned to the standard FMRIB58 FA template using a nonlinear registration, and the normalized FA images were then averaged to create a mean FA image. The mean FA image was thinned to create a FA skeleton representing WM tracts common to all patients. A threshold value of 0.2 was used to exclude adjacent gray matter or cerebrospinal fluid voxels.

A volume of tissue activated (VTA) was generated using the StimVison toolbox using patients' specific chronic stimulation settings (i.e., 130 Hz, 3.5V, 90 μs). White matter tracts passing through VTA were extracted in each subject using the Xtract toolbox in FSL and then averaged to generate a white matter tract mask that represents common activation pathways of all five subjects. Three white matter masks, including forceps minor (FM), cingulum bundle (CB), and uncinate fasciculus (UF), were included for the statistical analysis. Within the specific tracks of FA skeleton, Spearman's rank correlation between FA and two inferred transition times was performed to evaluate whether WM microstructure at baseline could predict the inferred transitions in states. The threshold was set at uncorrected p<0.05.

Statistical Analysis

Linear mixed models were used to test the association between SDCs and clinical assessment scores, and SDCs and facial expression classifier predictions with SDCs as the fixed factor and participants as the random factor. Models were fitted using the imertest package which uses a Sattherwaite approximation for degrees of freedom for ANOVA.

Second Methods

A cohort of TRD patients (e.g., such as those previously described) who received SCC DBS therapy and achieved high response rates (90% response, 70% remission) despite highly variable recovery trajectories and the need for complex clinical management. We derived an objective spectral discriminative component (SDC) that accurately captured clinically defined ‘sick’ and ‘stable response’ states, as well as responded to changes in DBS stimulation. In addition, the transition to reach the ‘stable response’ state identified from the SDC was correlated with (structural and functional) irregularities in the targeted white matter tracts and was further concordant with a data-driven analysis of the face. While moment-to-moment mood variations occur naturally, and short-lived behavioral effects are reliably produced with initial exposure to DBS, it is notable that the SDC behavior matches the clinical observation that sustained stable recovery requires weeks of ongoing chronic stimulation.

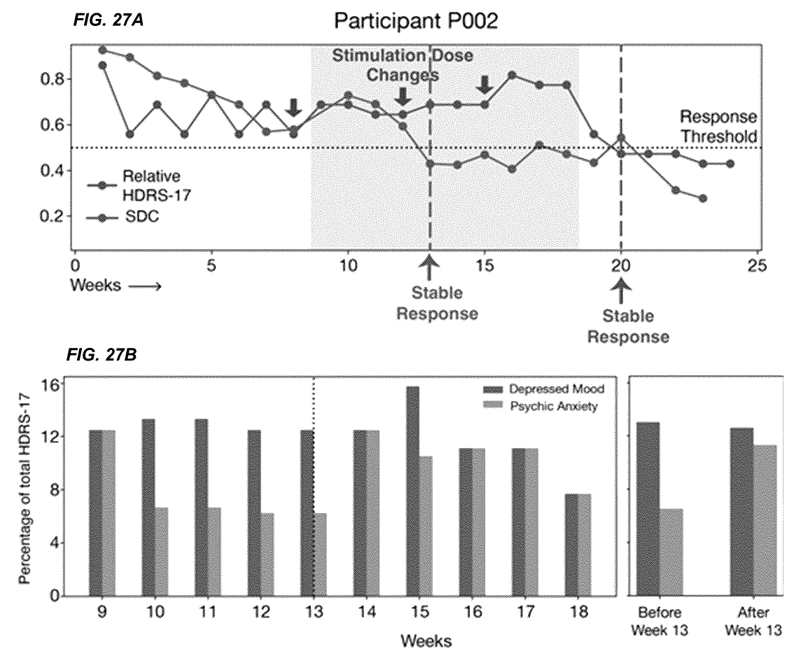

Our retrospective analysis of the relapsed responder further demonstrates the potential use and value of the SDC biomarker in a clinical setting. Specifically, the SDC predicted a relapse in this patient approximately four weeks before the structured interviews provided any indication of the pending clinical change. Conversely, we also observed a different participant (P003) where the SDC indicated a transition to stable recovery well before recovery was indicated by the HDRS-17. Further analysis of the individual HDRS-17 items revealed that the apparent mismatch of the HDRS-17 and the SDC was due to increasing anxiety symptoms without changes in core depression symptoms, a dissociation confirmed by clinical notes made by the study psychiatrist (e.g., as shown in FIG. 27). Thus, our observations suggest that the SDC can aid in distinguishing the two scenarios laid out in FIG. 2E, adding critical information to inform rational clinical management decisions. Replication in an independent cohort will provide additional specificity and sensitivity of the SDC necessary for implementation as a control signal in a ‘clinician-in-the-loop’ DBS approach.

Symptom improvement has been demonstrated when stimulating the right ventral capsule/ventral striatum (a different DBS target) based on changes in a different LFP marker (gamma-band power in the right amygdala) that correlated with acute fluctuations in mood and affect. However, it is not clear if this marker also captures a depressive state indicating stable recovery or only captures the short-term behavioral changes used to identify the marker. We derived the SDC based on a categorical definition of sustained recovery, thereby providing an explicit physiological signature discriminating between ‘sick’ and ‘well’ states. The SDC therefore tracks the totality of depression symptoms that define clinical improvement, is deliberately independent of transient mood fluctuations, and is consistent across all patients in the cohort (N=6). This evidence suggests that the proposed SDC marker tracks the depression syndrome instead of a particular idiosyncratic transient negative state and provides an objective measure suitable for clinical decision-making.

Multiple previous studies have also investigated acute SCC LFP dynamics with short term stimulation exposure or with resting state or emotional challenge experiments without stimulation exposure. All of these studies identify prominent (but not exclusive) beta band changes. Previous studies from our group demonstrate decreases in beta band power with brief bilateral intraoperative testing of the DTI-optimized contacts associated with later clinical response. Interestingly, the initial early decrease in beta band power observed within the first month of the observation phase in the current cohort is consistent with these previously reported acute stimulation effects. These initial decreases in beta band power in the current cohort were accompanied by clear decreases in symptom scores (18%), but were well below the threshold criteria for standard antidepressant clinical response (50% of baseline HDRS17) and typically required ongoing stimulation with voltage adjustments to achieve stable recovery. The eventual transition to an increase in beta and gamma band power only after chronic stimulation suggests that sustained, antidepressant responses are distinct from transient behavioral stimulation effects and thus are likely mediated by different mechanisms. Our findings support the broader hypothesis that beta band activity signals the establishment and maintenance of a status quo cognitive state. In this context, we posit that the early desynchronization of beta band activity with acute stimulation may correspond to release from the maladaptive state, which in turn, leads to enhancement of more flexible (e.g., adaptive) behavior reflected by increased HDRS variability. The subsequent increase in beta band activity with ongoing stimulation may thus signal the return of a new homeostatic set point after adaptation to chronic DBS, corresponding to the stable recovered state. One previous study identified that differences in 1/f slope of the right SCC LFP spectra partially captures changes in MADRS irrespective of the stability of the change suggesting a reorganization of excitation-inhibition balance.

The acute and chronic effects observed here with chronic DBS are similar to the effects observed with both rapid-acting and slow-acting antidepressants, particularly ketamine and selective serotonin reuptake inhibitors (SSRIs), respectively. Ketamine has been hypothesized to produce an antidepressant effect by inhibiting GABAergic interneurons leading to disinhibition of glutamatergic pyramidal neurons. In human EEG studies, ketamine administration has also been shown to acutely decrease beta oscillations. The early phase effects observed in our study may be mediated by a similar disinhibition. In contrast, the effect of SSRIs on 5-HT neural activity in the dorsal raphe nucleus (DRN, one of the downstream targets of SCC DBS) has been shown to change over time, with acute suppression followed by restoration over 2 weeks. Interestingly, chronic DBS has been shown to act on DRN neurons, restoring serotonergic pathways from DRN to limbic regions that include the ventromedial prefrontal cortex. Thus, the sustained increase in beta and gamma band powers with SCC DBS identified with our novel XAI approach may reflect a restoration of network function similar to this restoration of neuronal firing to produce a sustained antidepressant effect. Taken together, the neurophysiological effect of DBS may be an early disinhibition that produces an acute transient antidepressant effect followed by a restoration of the inhibitory control that results in stable recovery. This may also be reflected in observations of changes in clinical symptoms. In the early phase of treatment, changes are observed predominantly in negative mood and psychomotor slowing (including action intentionality), with progressive changes in anhedonia, neurovegetative and cognitive symptoms observed over time. However, the mechanism underlying a switch between the two effects is not clear.

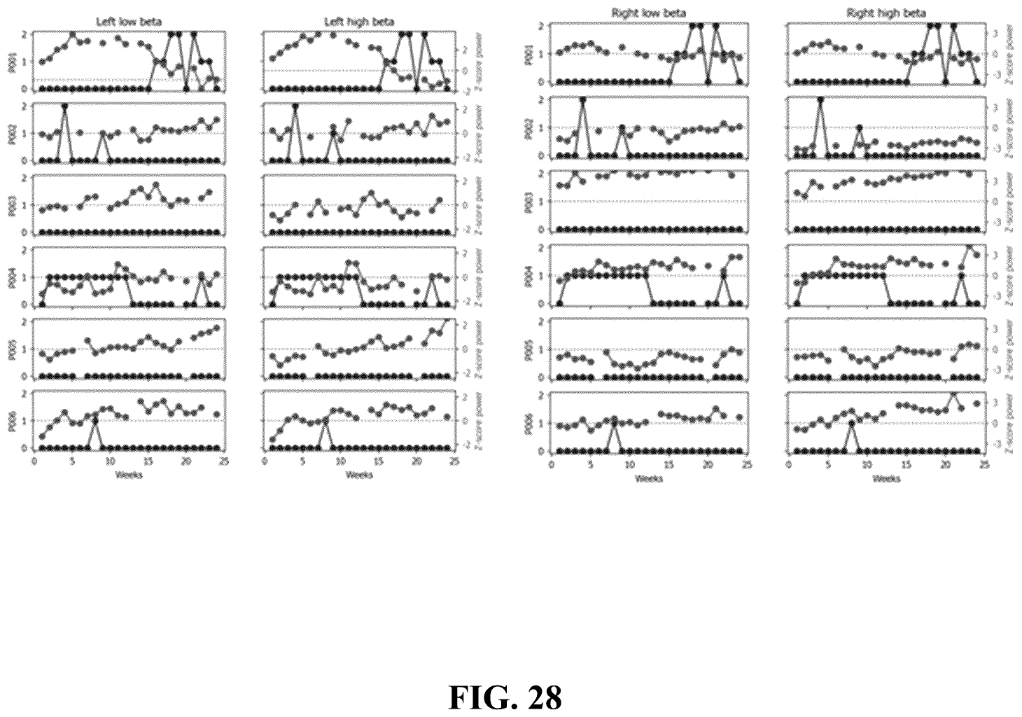

Beta band activity has emerged as an important marker of dysfunction across many studies investigating mood disorders. For example, beta band coherence between the amygdala and hippocampus was demonstrated to vary with short-term mood fluctuations. In a rodent model, beta band connectivity across multiple regions (including infralimbic cortex, a homolog of SCC) was found to reflect depressive symptoms and was engaged by optogenetic stimulation. Beta band functional connectivity between subgenual cingulate cortex and posterior cingulate cortex was implicated in ruminative behavior in patients in remission using scalp EEG. Interestingly, the different regions investigated in these studies constitute the targets of the white matter tracts being stimulated by SCC DBS. Thus, the beta band changes we observe may reflect network-wide changes across multiple regions. In addition, a computational model of DBS-induced recovery in the dynamics of the ventral cingulate cortex predicted the restoration of beta oscillations. While beta band abnormalities underlying bradykinesia in Parkinson's disease may be related to psychomotor slowness observed in depression, we did not find any evidence supporting this in our study (e.g., as shown in FIG. 28).

We found that the time required to achieve stable recovery among similarly treatment refractory patients was correlated with irregularities in the white matter tracts targeted by SCC DBS. This suggests that integrity of pathways within the network may be crucial to recovery and may mediate individual differences in recovery trajectory. Consistent with our results showing that increased RD and decreased FA (typically suggesting myelin loss) correlated with longer recovery times, previous evidence from postmortem, neuroimaging, and animal research suggests that depression is associated with myelin and oligodendroglia abnormalities. Furthermore, dACC FA is significantly associated with functional connectivity deficits between the stimulation target and MCC, which are directly connected via the cingulum bundle. Importantly, post-hoc analysis in nine subjects found a negative correlation between (structural and functional) white matter deficits and the number of lifetime depressive episodes. This result is consistent with a large depression cohort study that reported lower FA and higher RD with recurrent patients compared to one-episode patients, as well as previous studies relating the cumulative effects of depressive episodes on brain microstructure. While the cause and effect relationship of white matter damage, functional dysconnectivity and number of lifetime depressive episodes cannot be determined from these data, the location of the white matter findings within the cingulum bundle, uncinate fasciculus and forceps minor are situated to have significant impact on communication between the SCC and the default mode, salience, somatomotor and affective networks that mediate aspects of mood, arousal/interoception and psychomotor symptoms characteristic of major depression. Network reorganization may be a potential mechanism of the switch from acute to chronic response with SCC DBS, consistent with animal studies suggesting that chronic stimulation may lead to neuroplastic changes (including activation of oligodendroglial progenitor cells and new oligodendroglia), resulting in remyelination of targeted tracts or engagement of homeostatic plasticity mechanisms to produce long-term changes. The availability of new MRI-compatible implanted DBS pulse generators now enable direct measurements of structural and functional connectivity changes within the stimulated network over time to test these hypotheses. It is worth noting that the present imaging findings do not yet address TRD diagnostics or patient selection for SCC DBS, though clinical practice would certainly benefit from future work establishing rigorous predictors of patient outcomes.

Depression is characterized by multiple symptoms, many of which have long been known to be apparent on the face. We derived personalized facial expression patterns reflecting the discrete depression states in each individual. While there was clear overlap of the face action units that changed over time across subjects, the inability to derive a single sick/well classifier (either due to inherent variability or a limitation of the small sample size) meant that the model can only be used as a descriptive tool instead of a predictive tool to prospectively estimate the current depression state. A patient's appearance is a core component of a physician's clinical assessment during diagnosis and recovery, and the video analyses here provide a robust independent readout of these clinical impressions as a secondary confirmation of the brain biomarker. Furthermore, the specific nature of the face analysis yields insight into the connection between TRD symptoms and network pathologies. The common changes across subjects involve AUs previously linked to classic constructs of both sadness and happiness as well the EMG patterns of pain and despair as defined by Duchenne and Darwin in the 1860s. Importantly, the dominant bilaterally symmetrical AU changes in the upper face are consistent with our tractography-defined DBS target impacting the cingulum bundle, which projects to subregions of the facial nuclei that innervate the various muscles actuating the upper face (e.g., orbicularis oculi muscle and frontalis/corrugator muscular complex). Deficits in this pattern of facial movement (loss of mimetic facial expression, with preservation of volitional facial movement) is well described with lesions of the cingulum bundle. Furthermore, the dACC white matter lesion reported above is adjacent to the cingulate face region. That said, the upper face does not work in isolation, but the change pattern across all patients here is consistent with normalization of emotional rather than volitional facial movement. We note that volitional facial movements are moderated by M1 projections from the lateral cortex, which is a region not impacted directly by SCC DBS.

The current study has several limitations worth noting. First, the LFP analysis in the current study is limited to six of ten participants, with four participants unable to be included due to challenges associated with the first use of a prototype device (i.e., data artifacts in some devices, data collection protocol changes after initial SCC DBS pilot implantations). Hence, though the biomarker developed here shows consistency across the cohort, it may be biased by the small study sample. However, most modern machine learning approaches constantly update their models to improve generalization, thereby mitigating this limitation as more data becomes available. Second, the results presented are from LFP collected with stimulation turned OFF due to the presence of significant stimulation-related artifacts. However, while there is practical convenience to estimating a biomarker without interrupting therapeutic stimulation, the lack of negative clinical effects associated with relatively short SCC DBS discontinuation makes it feasible to calculate this biomarker during transient periods without the technical confound. Third, we have not explicitly modeled acute moment-moment distress in the current study, which would enhance the behavioral interpretability of our chronic depression marker. Future studies with increased data collection frequency will allow modeling of additional LFP signatures of potential clinical relevance. Finally, the analysis here is retrospective, leaving open questions about the exact use of the SDC in determining precise timing of optimal stimulation adjustments or the introduction of adjunct rehabilitative interventions such as cognitive behavioral therapy or mindfulness training

Participants and Clinical Assessments

Ten subjects with treatment-resistant major depressive disorder were consecutively enrolled in an experimental trial using a prototype deep brain stimulation device that allowed collection of local field potentials from the stimulation site (ClinicalTrials.gov Identifier NCT01984710). Participant characteristics are provided in table 4. All patients provided written informed consent to participate in the study. The protocol was in accordance with the Declaration of Helsinki. The protocol was approved by the Emory University Institutional Review Board and the US Food and Drug Administration under a physician-sponsored Investigational Device Exemption (IDE G130107) and is monitored by the Emory University Department of Psychiatry and Behavioral Sciences Data and Safety Monitoring Board. Clinical symptom severity was assessed by an independent rater using the 17-item Hamilton Depression Rating Scale (HDRS-17), Montgomery-Asberg Depression Rating Scale (MADRS), and self-reported Beck Depression Inventory (BDI) during weekly visits to the laboratory among other behavioral scales. Patients met weekly with the study psychiatrist who could make stimulation adjustments (increasing voltage by 0.5 V bilaterally) using a combination of HDRS-17 changes relative to the previous week and their clinical judgment. Following established criteria, a decrease in HDRS-17 scores greater than 50% of the presurgical average was set as the threshold for ‘response’. Remission was defined as a HDRS-17<8, and MADRS<10. Relative HDRS-17 and relative MADRS were computed as proportions of the presurgical average of HDRS-17 and MADRS, respectively.

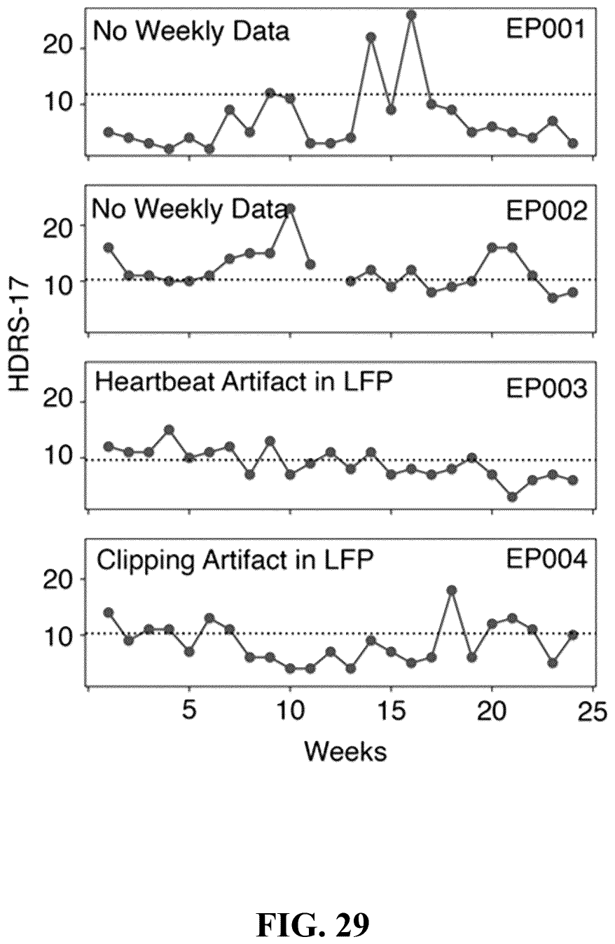

We report analysis of local field potentials (LFPs) from 6 participants listed in table 5 during a period of 6 months from the initiation of DBS therapy. Two participants were excluded from analysis as they had LFP data distorted by an amplifier clipping artifact (one participant) or heartbeat artifacts (one participant). Both of these participants were responders (>50% decrease in HDRS-17 from presurgical baseline) and one of them achieved remission (HDRS-17<8). The weekly trajectories of the excluded participants were not qualitatively different from the participants included in the study as shown in FIG. 29.

TABLE 4
Participant characteristics

| Characteristics | Mean (S.D) or Count | |
|---|---|---|
| Age at surgery | 49.40 | (11.2) |
| Sex | Female (7), Male (4) | |
| Employed at time of surgery | 2 of 10 | |
| Baseline HDRS-17 | 22.3 | (1.64) |
| Duration of current episode (months) | 47.3 | (44.03) |
| Age at first depressive episode (years) | 26.6 | (10.48) |
| Number of depressive episodes | 3.3 | (1.06) |

TABLE 5
Participant Demographics and Clinical History

| | | | Presurgery | | | | | |
|---|---|---|---|---|---|---|---|---|
| | Age at | | HDRS-17 | HDRS-17 | Stimulation | Stimulation | Number of | |
| | time of | | (mean | at the end | voltage at | voltage at | stimulation | Reason |
| Participant | surgery | | across 4 | of study | the start | end of | amplitude | for |
| ID | (years) | Sex | weeks) | (Week 24) | of study | study | changes | exclusion |
| P001 | 67 | F | 23.25 | 15 | 3.0 V | 4.5 V | 3 | N/A |
| P002 | 58 | F | 23.5 | 7 | 3.5 V | 4.0 V | 1 | N/A |
| P003 | 56 | F | 23.25 | 10 | 3.5 V | 5.0 V | 3 | N/A |
| P004 | 38 | M | 22.75 | 4 | 3.5 V | 4.5 V | 2 | N/A |
| P005 | 44 | F | 24.75 | 4 | 3.5 V | 3.5 V | 0 | N/A |
| P006 | 28 | M | 21.75 | 6 | 3.5 V | 4.5 V | 2 | N/A |
| EP001 | 45 | F | 23.5 | 3 | 3.5 V | 4.0 V | 1 | Weekly |
| | | | | | | | | LFP not |
| | | | | | | | | collected |
| EP002 | 43 | F | 20.5 | 8 | 3.5 V | 4.0 V | 1 | Weekly |
| | | | | | | | | LFP not |
| | | | | | | | | collected |
| EP003 | 60 | M | 19.25 | 0 | 3.5 V | 4.0 V | 1 | LFP |
| | | | | 6 | | | | contaminated |
| | | | | | | | | with |
| | | | | | | | | heartbeat |
| | | | | | | | | artifact |
| EP004 | 53 | M | 20.5 | 10 | 3.5 V | 4.0 V | 1 | LFP |
| | | | | | | | | affected |
| | | | | | | | | by |
| | | | | | | | | amplifier |
| | | | | | | | | clipping |
| | | | | | | | | artifact |

Subcallosal Cingulate Cortex Deep Brain Stimulation (DBS), Initial Settings, and Dose Adjustment.

Bilateral electrode array leads (e.g., such as 3387) were implanted in each participant, one in each SCC (FIG. 2A) as determined from tractography. A connectome-based targeting approach was used to identify targets that intersect four white matter pathways—forceps minor, cingulum bundle, uncinate fasciculus, and frontostriatal fibers (FIG. 2B). Stimulation was delivered using a voltage-controlled pulse generator Activa PC+S which also served as the local field potential acquisition system (Medtronic, Minneapolis, MN). DBS therapy started at least 30 days after the implantation surgery to allow for recovery from surgery. Therapy consisted of bilateral monopolar stimulation on a single contact per hemisphere at 130 Hz with 90 las pulse width. Stimulation amplitude was initially set at 3.5 V for all participants except P001. The initial amplitude for P003 was set at 3.0 V as the participant's symptoms were below the remission threshold at the beginning of the observation phase. During the observation phase, location, pulse width, and stimulation frequency remained unchanged. Dose was increased in steps of 0.5 V at unspecified intervals based on the study clinician's (PRP/AC) assessment of patient progress as described above. The initial stimulation voltage, stimulation voltage at the end of the 6-month study period, and the number of times stimulation voltage was changed in each participant are listed in table 4.

Local Field Potential (LFP) Recordings and Extraction of Spectral Features.

Local field potentials were acquired at a sampling rate of 422 Hz using Medtronic Activa PC+S system as differential recording from electrode contacts on either side of the stimulation contact to allow for common-mode rejection of noise as well as stimulation-related artifacts. LFPs were acquired weekly during the observation phase in a single 15-minute session in the laboratory. Each session consisted of two segments of approximately 7.5 minutes each—one with stimulation turned ON and the other with stimulation turned OFF. Only the segments with stimulation turned OFF were included in the analysis as the presence of stimulation-related artifacts precluded functional connectivity and cross-frequency coupling analyses. The first 10 seconds of the stimulation OFF period was discarded due to the presence of stimulation offset artifact (a slowly decaying signal reaching baseline). In addition, periods during which amplifier switching artifacts (presence of spike-like artifacts) were present were discarded. Finally, device-related frequency-drift artifacts were observed in beta and gamma band in a subset of the recordings of some participants. A robust PCA approach separated the device-related artifact into sparse components while the low-rank component contained the neural signals and was used in further analysis.

All LFP analyses were performed using custom-written scripts in Python (v3.6) and Matlab (R2018b). LFP recorded within a session was divided into 10-second segments from which spectral power, coherence and phase-amplitude coupling were estimated. Spectral power and magnitude-squared coherence were estimated using the python library Nitime's multi-taper fast Fourier transform approach with an adaptive procedure for setting the weights of tapers. Spectral power and coherence in canonical frequency bands (Delta: 1-4 Hz, Theta: 4-8 Hz, Alpha: 8-13 Hz, Low Beta: 13-20 Hz, High Beta: 20-30 Hz, Gamma 30-40 Hz) were then extracted as features for classification. The upper limit of the gamma band was restricted to 40 Hz instead of 50 Hz due to the presence of device-related artifacts in the range of 40-50 Hz.

Phase-amplitude coupling (PAC) was estimated using, for example, the PACtools python library. The algorithm was used to compute the coupling between low frequency (1-15 Hz) phase and high frequency (15-45 Hz) amplitude. Comodulograms were visually inspected to identify PAC regions of interest and PAC values between delta band (1.5-3 Hz) and high-beta/gamma band (20-35 Hz) were extracted as features. This procedure was followed to restrict the dimensionality of the features for the classifier, as including all the possible interactions would have considerably increased the feature set size. Thus, the overall dimensionality of the feature set was 20 (6 spectral features per hemisphere, 6 coherence features, 1 PAC feature per hemisphere).

Classification of LFP Features and Inferring the Spectral Discriminative Component (SDC)

Neural network models were used to classify LFP features using PyTorch. The parameters for the neural network models are listed in table 6. LFP spectral features were individually scaled between 0 and 1 as a pre-processing step. A 5-fold leave-one-out cross-validation was performed at the subject level to ensure generalizability. Models were fitted using LFP features from 4 out of 5 participants while the features from the 5th participant served as the test set. This procedure was repeated 5 times such that features from all 5 participants served as a test case.

TABLE 6
Parameters of Neural Network Classifier

| | |
|---|---|
| Architecture | 20 × 64 × 1 |
| Activation function | Rectified Linear Unit (ReLU) |
| Loss function | Binary cross-entropy |
| Learning rate | 0.001 |
| Optimizer | Adam |

We use the generative causal explanation (GCE) framework to identify interpretable features in the data that are determinative of the classifier's output. Conceptually, GCE can be thought of as a form of dimensionality reduction in which only a subset of the low-dimensional representation has a causal impact on the classifier output (e.g., as shown in FIG. 2D). This partitioning of the low-dimensional representation into classifier-relevant and classifier-irrelevant dimensions is accomplished by augmenting the objective of an autoencoder with a mutual information term that encourages a portion of the low-dimensional representation to influence the classifier output. We call the subset of dimensions in the low-dimensional representation that are relevant to the classifier's output the “discriminative components,” and the subset of the dimensions that contribute to representing the data but do not affect the classifier's output the “non-discriminative components.”

In the present work, the GCE was implemented using two separate networks: a feature compression network that maps the data from the feature space to the low-dimensional latent space, and a feature reconstruction network that reconstructs the feature space data from the latent components (e.g., as shown in FIG. 2D). The low-dimensional latent components were termed the spectral discriminative components (SDC) in one dimension and spectral non-discriminative components (SNDCs) in the remaining dimensions, based on the choice of parameters of GCE. The networks were trained to maximize the similarity of the reconstructed data and the true data using a loss function commonly used in variational auto-encoders as well as the information flow from the SDC to classifier output using a loss function. The GCE was trained with features extracted from LFP collected during the first month and last month of therapy in all participants and a classifier trained on the same data. Information flow from discriminative components to classifier output was higher than that of non-discriminative components, indicating that the SDC captures the features that determine the classifier output (e.g., as shown in FIG. 18A). A leave-one-out cross-validation was performed to make sure the model did not overfit. Briefly, GCE was trained on 4 out of the 5 participants and used to reconstruct the data of the 5th participant which was then used to evaluate the classifier's performance. This procedure was repeated 5 times leaving a unique participant's data out in each fold. The classifier's performance was comparable to the original data (e.g., as shown in FIG. 18B). In addition, to verify if only the discriminative component affected the classifier prediction, one of the components was randomized with other components unaffected and the classifier performance on the reconstructed data was evaluated. The entire procedure was performed in a leave-one-out fashion as described before. The performance of the classifier was affected when the discriminative component was randomized but not when the non-discriminative components were randomized verifying our design requirements. The reconstruction performance was evaluated by i) verifying that classification performance of a neural network classifier trained on the reconstructed data matched the performance of the classifier trained on the original data and ii) training a separate neural network classifier with original data and testing on the reconstructed data. In both cases the performance for the classifiers were comparable to the original classifier (Case (i) AUC=0.8, Case (ii) AUC=0.89±0.03; (e.g., as shown in FIG. 18B) suggesting the reconstruction captured the salient features of the original data. The parameters of the networks are listed in table 7.

TABLE 7
Parameters of GCE

| | Feature | Feature |
|---|---|---|
| | Compression | Reconstruction |
| | Network | Network |
| Architecture | 20 × 512 × 5 | 5 × 512 × 20 |
| Activation function | ReLU | ReLU |

| | |
|---|---|
| Learning rate | 0.0005 |
| Discriminative dimension | 1 |
| Non-Discriminative dimension | 3 |
| lambda | 0.1 |

The trained feature compression network was used to infer discriminative components of the LFP collected during months 2-5. LFP spectral features, computed in 10-second segments, were min-max scaled to the training set (LFP features from months 1 and 6) and projected through the feature compression network to infer discriminative and non-discriminative components. The SDC was transformed to probability of belonging to the ‘sick’ state as described in equation below as this allowed the SDC to be compared directly against the face classifier output. The SDC was then averaged across the 10-second segments within a week:

p_{sdc}=n(SDC_{sick}≤SDC)/(n(SDC_{sick})+n(SDC_{stable response})).

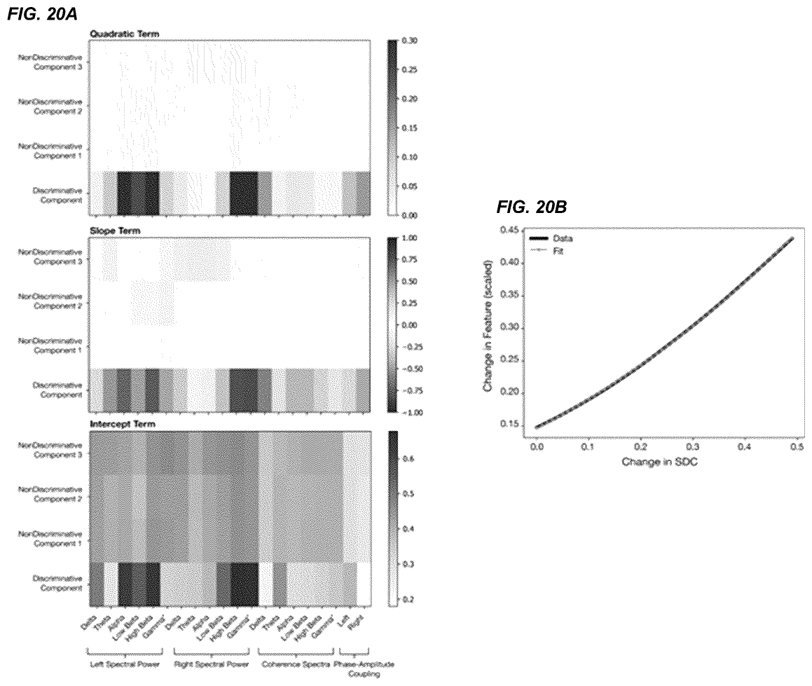

To map what features correspond to the SDC and SNDCs, the component values were varied in the latent space and passed through the feature reconstruction network. The resulting changes in the features were fit with second-order polynomials and the magnitude of the coefficients served as an indicator of feature change between weeks 1-4 and weeks 21-24 (e.g., as shown in FIG. 20A). Since the slope term captured most of the change (e.g., as shown in FIG. 20B), it was used as a measure of the features underlying the SDC.

Identifying Facial Expression Correlates of Behavioral Change and Decoding Face Classifier Output

In addition to clinical assessments, behavioral changes were estimated from facial expressions extracted from weekly videos of participants collected during the weekly psychiatric clinical interviews where LFPs were recorded and DBS management including dose changes were determined. Videos were recorded using a static, tripod-mounted video camera recording at 30 frames per second. The sessions were approximately thirty minutes long.

Videos were partitioned into 5-minute windows for feature generation with the remainders discarded. Each window was processed with the Openface facial behavior analysis toolkit V2.0. This open-source software produces presence, intensity, and confidence estimations for 18 facial action units, eye gaze, and head pose vectors, as well as 68 facial landmark positions for each frame. The 30 Hz frame rate was sufficiently granular to yield a temporal resolution to capture micro expressions (<0.5-second duration) as well as macro expressions (0.5 to 4 seconds). Data from frames with less than 93% confidence was discarded. The most common reason for discarding frames was the obstruction of the subjects' faces by their hands. From these first-order features, we generated second-order features consisting of envelope metrics (mean, median, quantiles, skew, kurtosis, variance) and covariance between features. From gaze and pose vectors we generated velocity, acceleration, jerk, and their envelope metrics. This processing was implemented in python resulting in 1073 features overall.

Using the same rationale as for the LFP classification, the facial expression features most differentially expressed between the ‘sick’ (weeks 1-4) and ‘stable response’ (weeks 21-24) states were identified using a paired two tailed t-test and used as input to train binary classifiers for each subject. For unbalanced sample sets due to sparse recordings, SMOTE was used to oversample the minority class. A logistic regression classifier with 10-fold cross-validation was implemented in the python sklearn library to discriminate the ‘sick’ from ‘stable response’ state for each subject. Following this, the trained classifiers were evaluated on the samples from the intermediate period to get the probability of being in the ‘sick’ state. The classifier predictions were termed ‘face classifier outputs’ and served as another behavioral marker to track response during ongoing DBS.

Identifying Transitions to Stable Response

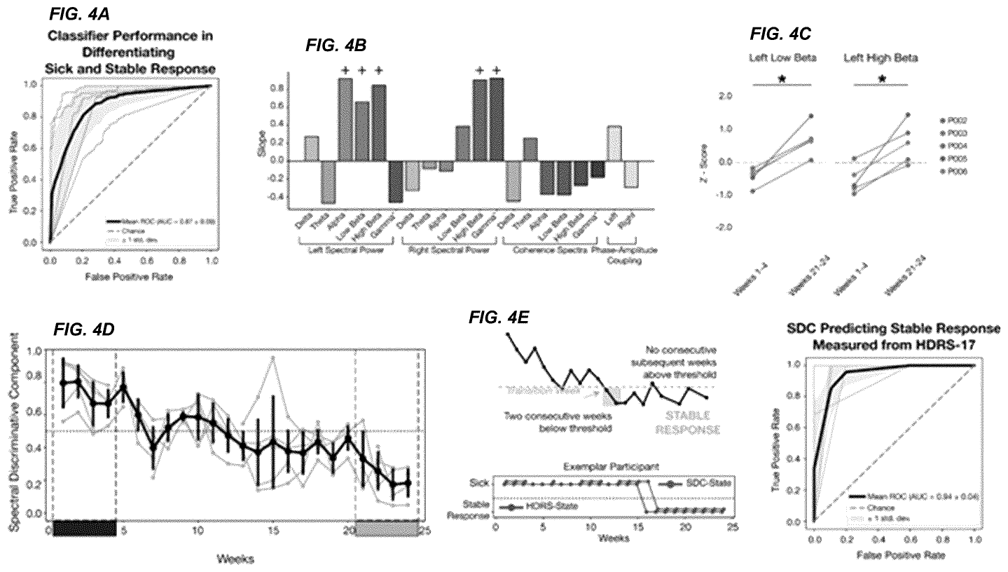

Patients receiving chronic therapeutic SCC DBS have been observed to show a characteristic response trajectory marked by a transient period of increased behavioral reactivity and instability followed by an improvement in symptoms that is sustained and stable. We inferred the week at which each of the participants reached this ‘stable response’ state based on weekly changes in HDRS-17, the SDC or the face classifier output (e.g., as shown in FIG. 4E). The transition was defined as the first of two consecutive weeks when the participant's measure fell below a defined threshold and did not increase beyond the threshold for two or more weeks.

In the case of HDRS-17, the relative score which the ratio of the aggregate score to the average of the presurgical baseline scores was used to define the states. A threshold of 0.5, indicating a decrease of 50% from presurgical baseline, was used to follow widely accepted definition of clinical response. In the case of the SDC and the face classifier output, it is not clear what the exact thresholds that correspond to clinical response should be. We used receiver operating characteristic curve, which focuses on sensitivity and selectivity of discriminability instead of hard thresholds, to compare against HDRS-17. However, when compared against each other, it is possible to use the same thresholds as the values indicate probability of being in the ‘sick’ state. We used a more conservative threshold of 0.35 to identify the transition to stable response.

The concordance between the weeks of transition was evaluated using Kendall's tau metric which is a rank-based correlation measure. Kendall's tau reflects the similarity in the ranks of the transition weeks, i.e., do the participants who exhibit a transition in SDC early also exhibit a transition in face early and vice versa.

Image Acquisition and Processing

High-resolution structural T1-weighted (T1w) and diffusion-weighted images (DWI) were acquired on a 3T Siemens Tim Trio MRI scanner (Siemens Medical Solutions). T1w images were collected using a 3D magnetization-prepared rapid gradient-echo (MPRAGE) sequence with the following parameters: sagittal slice orientation; resolution=1.0 mm×1.0 mm×1.0 mm; repetition time (TR)=2600 ms; inversion time (TI)=900 ms; echo time (TE)=3.02 ms; flip angle=8°. DWI was acquired using single-shot spin-echo echo-planar imaging (EPI) sequence with the following parameters: 64 non-collinear directions with five non-diffusion weighted images (b0), b-value=1000 sec/mm2; number of slices=64; field of view=256×256 mm2; voxel size=2×2×2 mm3; TR=11300 ms; TE=90 ms. Additional full DWI data set with opposite phase encoding was also collected to compensate for the susceptibility-induced distortion.

All images were preprocessed using the FMRIB Software Library. T1w image was skull stripped and normalized to MNI152 template using fsl_anat toolbox. DWI data underwent distortion and motion collection using the Eddy toolbox and a local tensor fitting to calculate the FA map. Tract-Based Spatial Statistics (TBSS) processing was performed for the group analysis. Briefly, individual FA images were aligned to the standard FMRIB58 FA template using a nonlinear registration, and the normalized FA images were then averaged to create a mean FA image. The mean FA image was thinned to create a FA skeleton representing WM tracts common to all patients. A threshold value of 0.2 was used to exclude adjacent gray matter or cerebrospinal fluid voxels. A similar process was done for the radial diffusivity (RD) and axonal diffusivity (AD).

A volume of tissue activated (VTA) was generated using the StimVison toolbox with patients' specific chronic stimulation settings (i.e., 130 Hz, 3.5V, 90 μs). White matter tracts passing through VTA were extracted in each subject using the Xtract toolbox in FSL and then averaged to generate a white matter tract mask that represents common activation pathways of all five subjects. Three white matter masks, including forceps minor (FM), cingulum bundle (CB), and uncinate fasciculus (UF), were included for the statistical analysis. Within the specific tracks of FA skeleton, Spearman's rank correlation between white matter integrity measures (FA, RD, and AD) and the inferred transition times was performed to evaluate whether WM microstructure at baseline could predict the inferred transitions in states.

To further explore the relationship between altered white matter microstructures/abnormal brain activity and DBS recovery trajectory, post-hoc correlation analyses were conducted in the identified brain regions from the correlation analysis of transition times with imaging using all ten subjects. Briefly, Spearman's rank correlation analysis (age and gender controlled) was performed between baseline white matter integrity (FA) and depression clinical features, including depression severity (HamD), duration of current episode, the number of episodes in a lifetime, and length of illness (duration between on-set and surgery). In addition, the same analyses were done for the resting-state functional connectivity using the bilateral SCC seeds.

Statistical Analysis

Hypothesis testing of changes in HDRS-17, SDC and individual features was performed using a one-sided Wilcoxon signed rank test. The non-parametric test was chosen to account for the small sample size and inability to test for normality. The small sample size of the current study does not have sufficient power to test statistical significance at 0.05 in a two-sided test even when the direction of changes are readily apparent. Therefore, we used a one-sided test with a threshold of 0.05 and also confirmed statistical significance in two-sided test with a relaxed threshold of 0.1. Linear mixed models were used to test the association between the SDC and clinical assessment scores, and the SDC and face classifier output (with the SDC as the fixed factor and participants as the random factor). Models were fitted using the ‘lmertest’ package which uses a Sattherwaite approximation for degrees of freedom for ANOVA. The threshold was set at uncorrected p<0.05 for all correlation analyses between imaging and the SDC.

Results

SCC DBS is Effective in Treating Depression Symptoms

The study cohort consisted of 10 consecutively recruited participants who were implanted with an experimental DBS implanted pulse generator (IPG) that served both stimulation and recording functions. DBS leads were implanted at the intersection of 4 major white matter pathways (as shown in FIG. 2A, 2B) identified from earlier studies. All participants met study inclusion criteria before implantation with a minimum depression severity HDRS-17 score equal or higher than 20 (Table 1). Stimulation was turned ON following a 4-week post-surgery recovery phase, and the primary endpoint of the study was defined as the HDRS-17 score at 24 weeks of chronic SCC DBS. At a cohort level, participants experienced a significant reduction in HDRS-17 score from pre-surgery baseline with a mean HDRS-17 of 23.21 (SD 0.89), to the end of the 24-week observation phase with a mean HDRS-17 of 7.67 (SD 3.86). At an individual level, 9 out of 10 participants were deemed to be responders (greater than 50% decrease in HDRS-17) and 7 out of 10 were deemed to be in remission (HDRS-17 less than 8). Despite the consistent clinical outcomes at the 24-week endpoint, individual patients showed variable recovery trajectories, with some achieving clinical response much earlier than others (as shown in FIG. 2C).

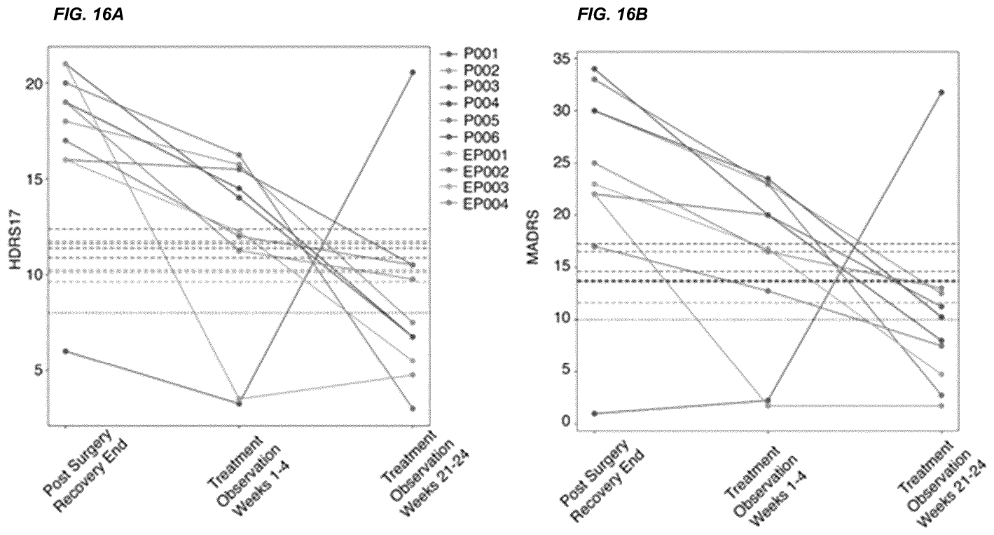

Chronic electrophysiological data for analyses was available for 6 of the 10 subjects. Of these subjects, five of the six demonstrated a typical response trajectory (‘typical responders’). The five participants entered the 24-week observation phase with a mean HDRS-17 of 18.80 (SD 1.72) reflecting a mean decrease of 4.4 (SD 2.15) following surgery and intraoperative stimulation. After four weeks of chronic stimulation, these ‘typical responders’ experienced a further decrease with a mean HDRS-17 of 15.20 (SD 0.83) (mean decrease 3.6, paired one-tailed Wilcoxon signed rank test, p=0.03), and in weeks 21 to 24 their mean HDRS-17 was 6.92 (SD 2.39) (e.g., as shown in FIG. 16). The difference in HDRS-17 between the first 4 weeks and the last 4 weeks was statistically significant (mean decrease 8.3, paired one-tailed Wilcoxon signed rank test, p=0.03). At the end of 24 weeks, these five participants reached clinical responder status, and four out of the five participants achieved remission. Based on the weekly HDRS-17 scores, all participants were considered to be in a ‘sick’ state during the first four weeks and in a ‘stable response’ state the last four weeks of this period.

LFP Classifier Identifies the Difference in SCC Dynamics Between the ‘Sick’ and ‘Stable Response’ State

We extracted spectral features from LFP recorded with stimulation turned OFF for the classification of ‘sick’ vs. ‘stable response’ (i.e., the first 4 weeks and the last 4 weeks of the 24 week observation period) in the typical responders. A neural network classifier (with leave-one-participant-out cross-validation) was able to distinguish the ‘sick’ and ‘stable response’ states (AUROC: 0.87±0.09; as shown in FIG. 4A) in the 5 typical responders, suggesting recovery from depression is reflected in similar electrophysiological changes across participants. We then trained a generative causal explainer (GCE) to identify the spectral discriminative component (SDC), which is a low-dimensional latent representation of the spectral features that collectively capture the difference between the ‘sick’ and ‘stable response’ states as determined by the neural network classifier. Thus, the SDC serves as an LFP marker reflecting the status relative to binary depressive/recovered states, with higher values indicating the ‘sick’ state and lower values indicating the ‘stable response’ state.

DBS Acutely Suppresses but Chronically Enhances SCC Beta Band Power

We used the slope of the joint changes in LFP features when the SDC was varied to identify the concurrent spectral features that exhibited the most changes when patients transitioned from ‘sick’ to ‘stable response’ (e.g., as shown in FIG. 4B). A positive slope indicates an increase in the feature's magnitude when the SDC changed from the ‘sick’ state to ‘stable response’ state while a negative slope indicates a decrease in the feature's magnitude. Changes in the SDC resulted in changes in many spectral features simultaneously, with the largest changes observed in left alpha (8-13 Hz), left low beta (13-20 Hz), left high beta (20-30 Hz), right high beta, and right gamma band power (30-40 Hz). All of these features exhibited an increase suggesting the difference between ‘sick’ and ‘stable response’ states is driven by bilateral increase in beta/gamma power in SCC. As a secondary confirmation, a similar subset of features was identified to be important for classification using a clustering-based permutation feature importance method (e.g., as shown in FIG. 10).

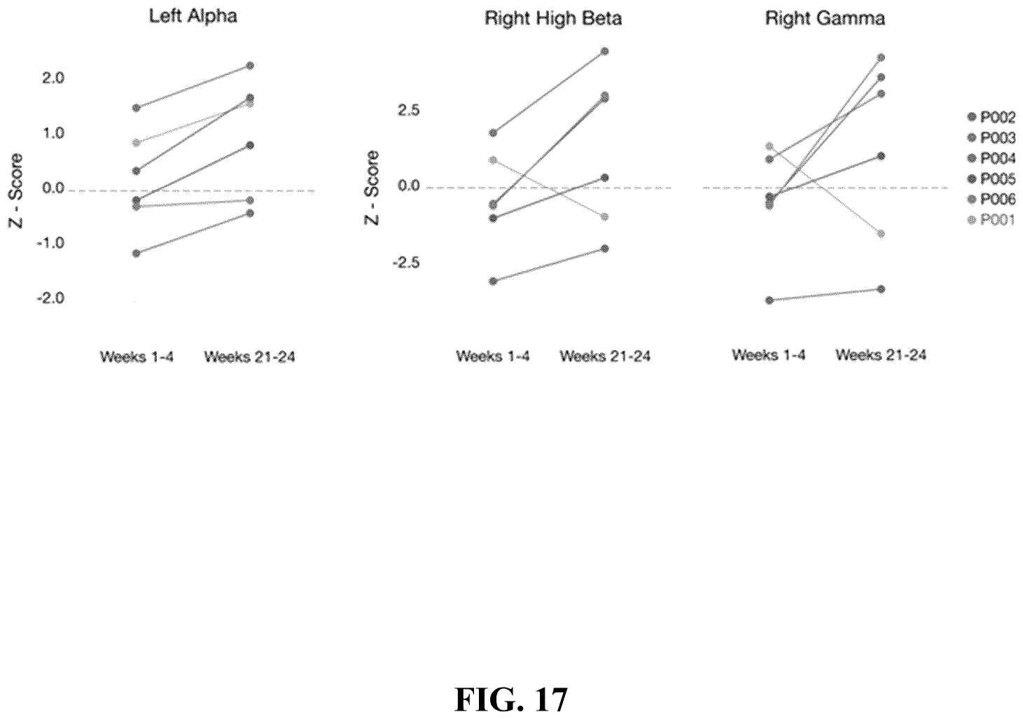

While the identified features (especially beta band power) have been previously reported to respond to stimulation in acute stimulation experiments, the current longitudinal analysis reports the opposite change pattern. Specifically, acute intraoperative SCC stimulation has been shown to decrease beta band power, whereas chronic stimulation here promotes sustained increases in beta band power. To directly compare findings here to these previous studies, we computed the beta band power across the 24 week observation phase relative to the last week of the 4-week post-surgery recovery phase (when stimulation remained OFF). Relative to the post-surgery OFF baseline, left low beta band power (13-20 Hz) was lower in the early phase of active treatment (week 1-4 Stim ON) (Wilcoxon signed rank test, p=0.031) and higher in the late phase (week 21-24 Stim ON) (Wilcoxon signed rank test, p=0.031) in all five typical responders (e.g., as shown in FIG. 4C). The difference between the early changes and the late changes was also statistically significant (paired one tailed Wilcoxon signed rank test, p=0.031). A similar difference between the early and late changes was observed in left high beta band power (p=0.031), although the early treatment decrease and late treatment increase were not statistically significant (p=0.062). This indicates that while the early effect of stimulation is consistent with the acute effects observed in previous studies, the long-term effect is distinct and in the opposite direction. While other bands with significant longitudinal changes (captured by the SDC) exhibit an increase from weeks 1-4 to weeks 21-24, only the low beta band activity exhibits the differential response of acute decrease followed by increase with chronic stimulation (e.g., as shown in FIG. 17).

The SDC Tracks Progress to Stable Response

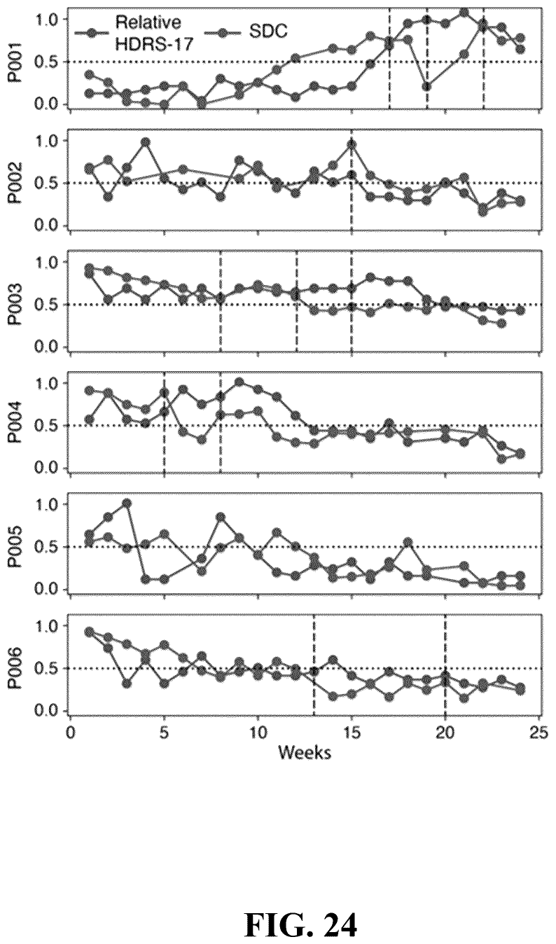

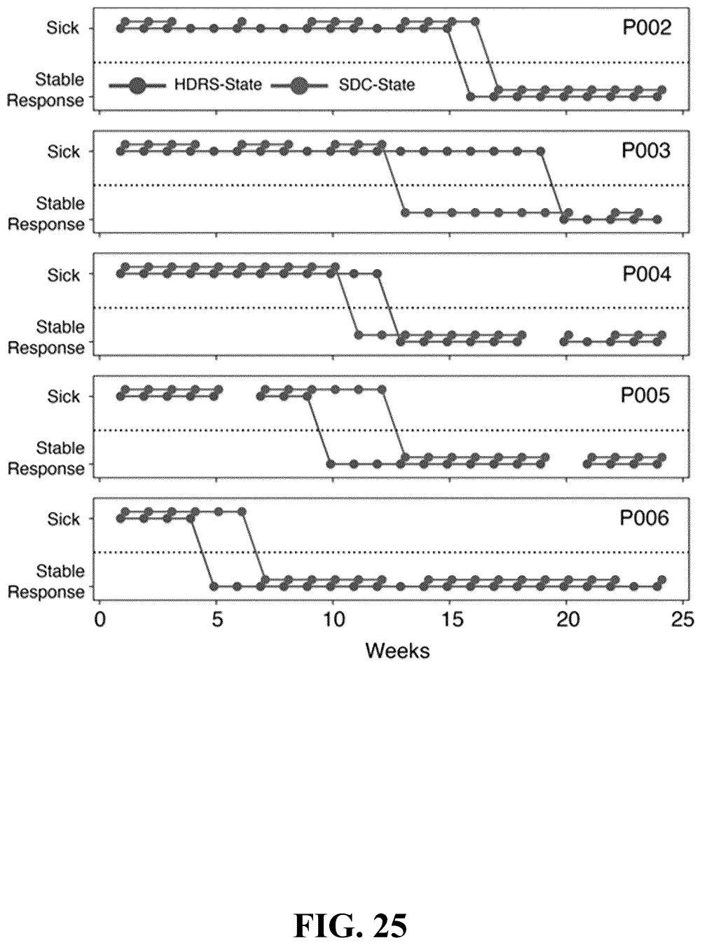

We computed the SDC for the intermediate period (weeks 5-20) to estimate the trajectory of LFP changes from the ‘sick’ state to the ‘stable response’ state in all patients (e.g., as shown in FIG. 4D and FIG. 24). To verify if the SDC indeed tracked depressive symptoms, we compared the depressive state estimated from the SDC against the state derived from HDRS-17. We further define ‘stable response’ as the occurrence of two or more consecutive weeks of therapeutic response, followed by the absence of a subsequent loss of response (e.g., as shown in FIG. 4E). The time of stable response is taken (in retrospective analysis) to be the first week a patient reached this ‘stable response’ state. Thus, the participants are considered to be in the ‘sick’ state in the weeks preceding this time point, and in the ‘stable response’ state in the weeks following this time point. Employing analogous criteria on the SDC, we examined the ability of the electrophysiology marker to detect this sick/stable response state on a weekly basis, as shown in FIG. 4F, FIG. 24 with a receiver operating curve (ROC). When evaluated using the area under the curve (AUC) for each subject (e.g., as shown in FIG. 4G), we found this approach yields high accuracy; in weekly estimates the SDC-state matched the HDRS-state over 90% of the time (AUC 0.94±0.04), indicating that SDC significantly and reliably captures clinically meaningful depression states of the participants. FIG. 24 shows how well the state derived from SDC using a threshold of 0.5 tracks the state derived from HDRS-17.

Changes in Stimulation Voltage Engage the SDC

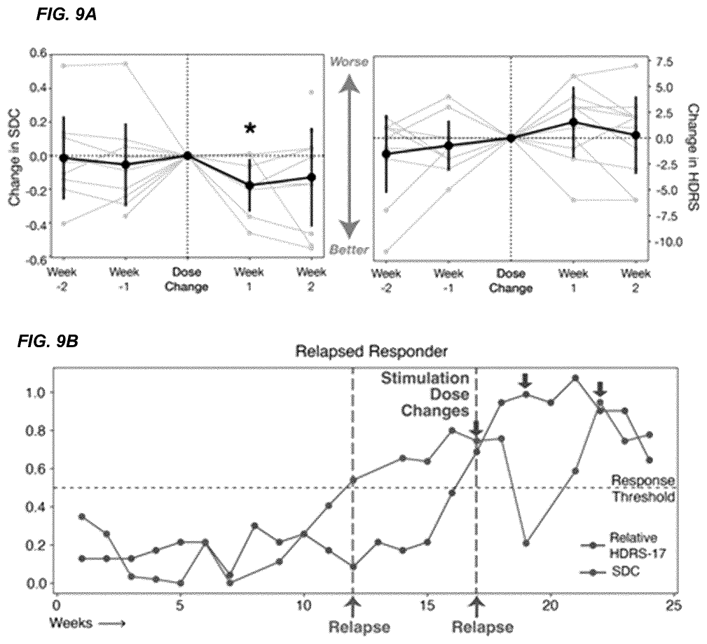

While all participants start at the same dose (a stimulation voltage of 3.5 V) at the beginning of the 24 week treatment protocol (except P001 who started at 3 V), the dose may be changed as deemed necessary by the study psychiatrist in increments of 0.5 V (Table 1). The weeks in which these changes were made varied across participants (range: 4 to 22 weeks after the beginning of therapeutic stimulation). This provides an opportunity to examine if the SDC is affected by DBS dose adjustments. We found that increases in stimulation voltage resulted in a decrease in the SDC (i.e., the LFP indicated progress toward the ‘well’ state) in the subsequent week (e.g., as shown in FIG. 9A) left; −0.177±0.111, 1 sample Wilcoxon signed-rank sum test p=0.039), suggesting that the LFP features that capture stable depression recovery are affected by stimulation voltage change. In contrast, the changes in voltage did not result in a consistent or significant change in HDRS-17 scores in the subsequent week (e.g., as shown in FIG. 9A) right, 1 sample Wilcoxon signed-rank test p=0.307). We also found that the changes observed 1 week after stimulation voltage change were statistically different from the changes observed 1 week after a random week when no stimulation voltage change was made (p=0.034) using a shuffle-based procedure (e.g., as shown in FIG. 10D).

The SDC Identifies Relapse in an Out-of-Sample Participant

To demonstrate the potential utility of the SDC in a clinical setting, we retrospectively analyzed LFP data from one participant (P001) whose data was not included in training the classifier or the GCE. Thus, this participant served as an out-of-sample validation data point for the SDC as a depression state biomarker. P001 experienced a clinical relapse after four months in remission. P001 started the active stimulation phase with low HDRS-17 scores (<8) and had a sudden and sustained worsening of symptoms such that they were deemed a non-responder by week 16 (e.g., as shown in FIG. 9 blue line). Using the SDC trained on the five typical responders (but not trained on P001), the SDC correctly captures this trend in P001 by indicating a response state followed by a sick state (red line). Interestingly, the SDC indicated a relapse from the brain signal (red arrow) over one month prior to the clinical relapse measured by the HDRS-17 (blue arrow), demonstrating that the brain biomarker could have predicted an impending instability and the need for earlier intervention before it was clinically apparent. In addition, dose increases (purple arrows) resulted in decreases in SDC but the effect did not persist until changes were made three times. Notably the final stable dose in this patient (4.5 V) after the 6-month study period was comparable to the average dose in the typical responders.

To demonstrate the similarity between HDRS-17 and the SDC in this out-of-sample participant, we compared the states indicated by HDRS-17 and the SDC. Since the therapeutic response was at the beginning of the observation phase, it is not possible to use the criteria described above for ‘stable response’. Yet, if we consider the two states as ‘sick’ and ‘response’ denoting a change in HDRS-17 of less than 50% decrease and greater than 50% decrease (respectively), we find that the SDC-state accurately predicts the HDRS-state 75% of the time over the 6 month treatment course (p=0.03, shuffle-based procedure).

White Matter Abnormality Correlates of Transition to ‘Stable Response’ State

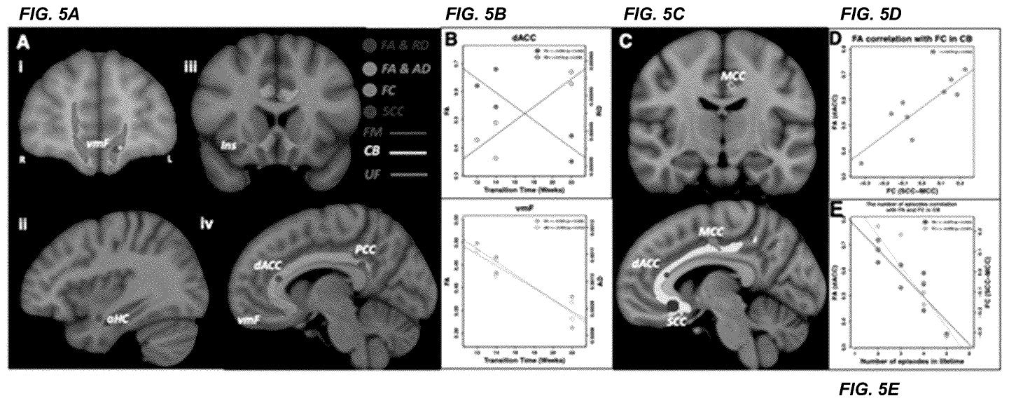

Previous studies have shown that incomplete white matter pathway activation impacts therapeutic outcomes in SCC DBS. We hypothesized here that functional and structural abnormalities in these pre-specified targeted white matter bundles may also influence the recovery trajectory, as inferred from the SDC. Using preoperative imaging, we found significant negative correlations between the weeks of transition to ‘stable response’ as identified from the SDC and white matter integrity, as indexed by both fractional anisotropy (FA) and radial diffusivity (RD). Regions having particularly significant correlations between structural integrity and time to recovery within the target network include the forceps minor, uncinate fasciculus, frontal-subcortical and cingulum bundles connecting the DBS target site to the ventromedial frontal (vmF) cortex, anterior hippocampus (aHc), insular (Ins), and dorsal anterior and posterior cingulate cortex (dACC and PCC), respectively (e.g., as shown in FIG. 5A, 5B). These findings suggest that white matter microstructure alterations within the underlying targeted brain network results in longer DBS treatment times to achieve a stable response. Specifically, the RD correlation with time to recovery provides evidence of baseline demyelination being a primary contributor to the white matter deficits that account for the variable time to recovery in patients.

In addition to a relationship between the stable SDC response time and white matter damage, we found a significant correlation of white matter abnormalities in the dACC to functional connectivity between the SCC and the midcingulate cortex (MCC) (p<0.05) (e.g., as shown in FIG. 5C, 5D). This correspondence indicates a relationship between functional properties within the target network and structural properties that account for a prospective notion of disease severity as indexed by time to recovery. Furthermore, when considering the whole cohort, we found a significant negative correlation of both dACC FA and SCC-MCC functional connectivity with the number of lifetime depressive episodes experienced by each individual prior to SCC DBS (n=9 subjects; one excluded due to artifact) (p<0.05) (e.g., as shown in FIG. 5E). This concordance suggests that structural and functional deficits in the target network are also related to a retrospective notion of disease severity as indexed by the individual patient's history of chronic depression.

The SDC Tracks Changes in Depressive Symptoms Captured by Facial Expressions

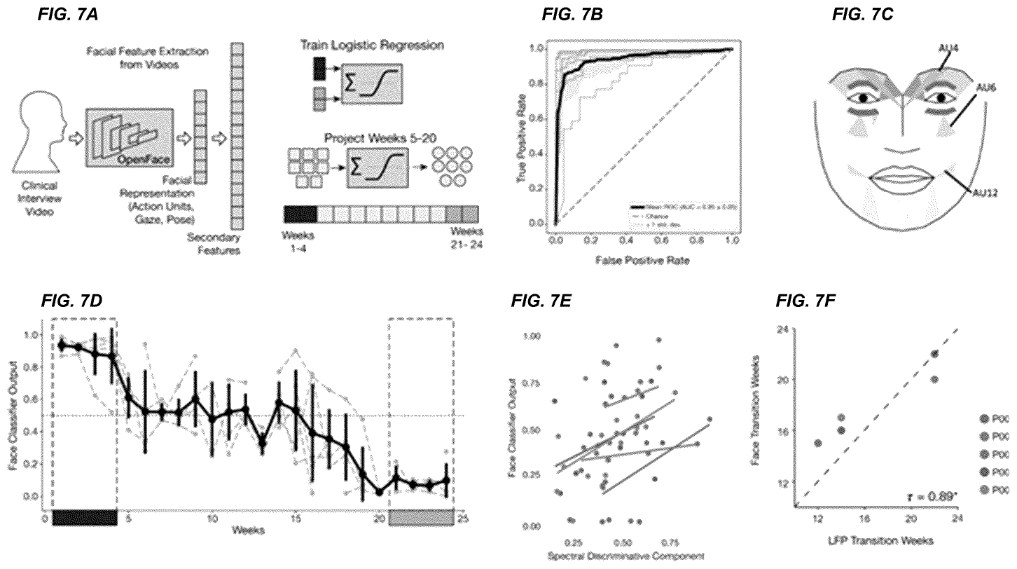

In addition to standardized clinical rating scales, we quantified behavioral improvement using changes in facial expression extracted from videos of weekly clinical interviews. The features comprised summary measures of facial movements including facial action units, eye gaze and head pose (e.g., as shown in FIG. 7A). Importantly, the features were not designed to be explicitly related to any specific emotion constructs (e.g., sadness). Similar to the LFP analyses (but entirely independent of the LFP data), we aimed to identify differences between the ‘sick’ and ‘stable response’ states using the facial features. Since there are considerable inter-individual differences in facial expressions independent of depression, we used an individualized classifier for each patient to distinguish ‘sick’ and ‘stable response’ time periods (in contrast to the single LFP classifier derived for the whole cohort). Logistic regression classifiers of facial expression features were able to classify ‘sick’ and ‘stable response’ states in each individual participant separately (AUROC 0.95±0.05), suggesting that there are individualized yet consistent differences between the ‘sick’ and ‘stable response’ states (e.g., as shown in FIG. 7B). While we found a common set of distinct features (action units 1 and 7 and pose) across all participants in the consensus map (e.g., as shown in FIG. 5C), there were also many features that distinguished ‘sick’ and ‘stable response’ states unique to each participant.

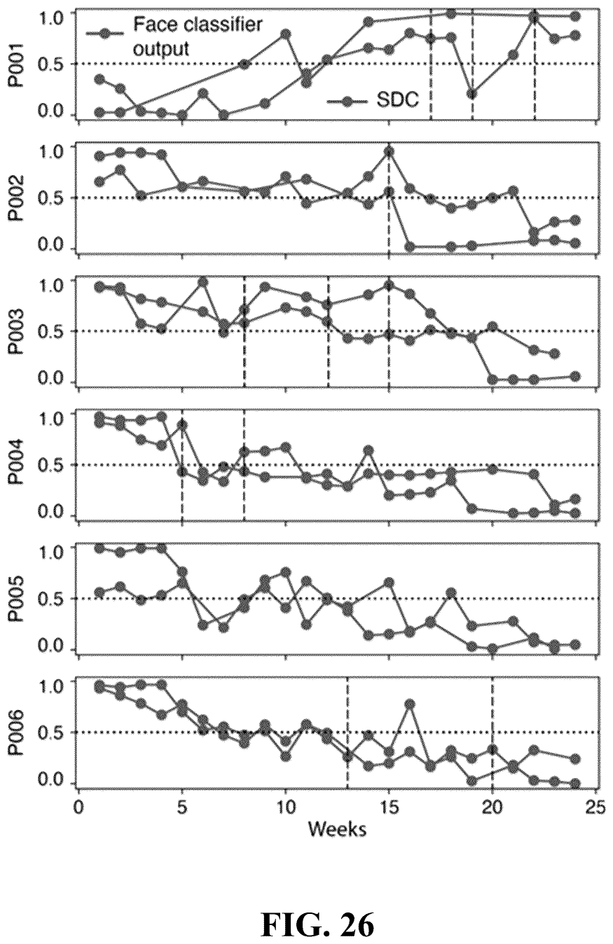

We then used these individual facial expression features extracted in the intermediate period (weeks 5-20) to obtain the classifier's prediction of disease state, which we termed face classifier output. As a secondary confirmation of the SDC biomarker, we compared the face classifier output to the SDC for each individual patient. We observed that the face classifier output's trajectory is both qualitatively similar to the corresponding participant's SDC trajectory (e.g., as shown in FIG. 7D, FIG. 26), and quantitatively we found a significant relationship between the face classifier output and the SDC (as shown in FIG. 7E; Linear mixed model, F(1, 51.74)=6.54, p=0.01). Next, we tested if the face classifier output captures the changes from ‘sick’ to ‘stable response’ that is observed in the SDC. The face classifier output and the SDC have the same normalized scale (unlike HDRS-17), meaning they are directly comparable. Using a strict threshold (0.35) to binarize these measures for direct comparison, we found that the transition weeks from the ‘sick’ state to the ‘stable response’ state inferred from the SDC and the face classifier output were concordant (as shown in FIG. 7F); Kendall's tau=0.89, p=0.04). Taken together, these results suggest that the SDC also accurately tracks changes in facial expressions accompanying recovery from depression.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims,

What is claimed is:

1. A method to assess major depressive disorder (MDD) disease state in a subject during the course of therapy, the method comprising:
receiving electrophysiological measurements for assessment; and
generating, using a neural network, a prediction of a disease state based on the electrophysical measurements.

2. A method characterizing a depression state of a subject during the course of therapy, the method comprising:
receiving electrophysiological measurements from a sensor associated with a brain of the subject that characterizes the depression state progression; and
generating the depression state based on the electrophysiological measurements.

3. The method or system of claim 2, wherein the characterization comprises the identification of at least one discrete disease state or the disease trajectory within at least one disease state.

4. A method comprising:
using electrophysiological signals as a biomarkers to assess MDD disease state in a subject during the course of therapy, characterize the progression of MDD in a subject during the course of therapy, and/or monitor, characterize, and/or assess discrete transitions in behavior during the course of therapy.

5. The method of claim 1, wherein the electrophysiological measurements are associated with neural stimulation.

6. The method of claim 5, wherein the neural stimulation is acute.

7. A method to track changes in facial feature between discrete disease states.

8. The method of claim 1, wherein a brain map is configured to predict transitions in brain states of a subject.

9. A system comprising:
one or more processors; and
a non-transitory computer-readable medium storing instructions that, when executed by the one or more processors, cause the one or more processors to perform operations including:
receiving electrophysiological measurements for assessment; and
generating, using a neural network, a prediction of a disease state based on the electrophysical measurements.

10. A non-transitory computer-readable medium storing instructions that, when executed by one or more processors, cause the one or more processors to perform operations including:
receiving electrophysiological measurements for assessment; and
generating, using a neural network, a prediction of a disease state based on the electrophysical measurements.

11. A method to assess major depressive disorder (MDD) disease state in a subject during the course of therapy, the method comprising:
receiving one or more facial features; and
generating, using a neural network, a prediction of a disease state based on the one or more facial features.

12. The method of claim 11, wherein the disease state comprises the identification of at a disease trajectory within at least one disease state.

13. The method of claim 11, wherein the one or more facial features are received before or after an instance of electrophysical therapy.
